(19) United States
(12) Patent Application Publication
(10) Pub. No.: US 2013/0236968 A1
(43) Pub. Date: Sep. 12, 2013

(54) MULTIFUNCTIONAL COPOLYMERS FOR NUCLEIC ACID DELIVERY

(71) Applicants: Muthiah Manoharan, Cambridge, MA (US); Kallanthottathil G. Rajeev, Cambridge, MA (US)

(72) Inventors: Muthiah Manoharan, Cambridge, MA (US); Kallanthottathil G. Rajeev, Cambridge, MA (US)

(73) Assignee: ALNYLAM PHARMACEUTICALS, INC., Cambridge, MA (US)

(21) Appl. No.: 13/805,991

(22) PCT Filed: Jun. 20, 2011
(86) PCT No.: PCT/US11/41062

§ 371 Date: Mar. 8, 2013

Related U.S. Application Data
(60) Provisional application No. 61/356,793, filed on Jun. 21, 2010.

(51) Int. Cl. C12N 15/113; C08F 222/38; C08F 224/00; C08F 228/06; C08F 226/06

(52) U.S. Cl. CPC C12N 15/113; C08F 228/06; C08F 226/06; C08F 224/00; C08F 222/38
USPC 435/375; 526/257; 526/265; 526/238.23; 526/284; 526/258

(57) ABSTRACT

The present invention relates to multifunctional polymers represented by the following formula:

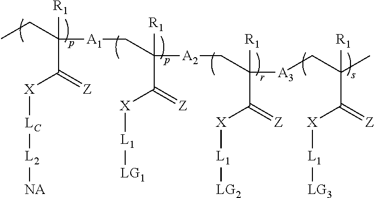

The invention further provides methods for their preparation and methods for site-specific delivery of nucleic acids by combining them with targeting ligands, endosomolytic ligands and/or PK modulator ligands.

### PRIORITY CLAIM

This application claims priority of U.S. Provisional Application No. 61/356,793, filed Jun. 21, 2010, the content of which is incorporated herein by reference in its entirety.

### BACKGROUND

RNA interference or “RNAi” is a term initially coined by Fire and co-workers to describe the observation that certain double-stranded RNA (dsRNA) can block gene expression when it is introduced into worms (Fire et al. (1998) Nature 391, 806-811). Short double-stranded interfering RNA (dsiRNA) directs gene-specific, post-transcriptional silencing in many organisms, including vertebrates, and has provided a new tool for studying gene function. RNAi may involve mRNA degradation.

Work in this field is typified by comparatively cumbersome approaches to delivery of dsiRNA to live mammals. E.g., McCaffrey et al. (Nature 418:38-39, 2002) demonstrated the use of dsiRNA to inhibit the expression of a luciferase reporter gene in mice. The dsiRNAs were administered by the method of hydrodynamic tail vein injections (in addition, inhibition appeared to depend on the injection of greater than 2 mg/kg dsiRNA). The inventors have discovered, inter alia, that the unwieldy methods typical of some reported work are not needed to provide effective amounts of dsiRNA to mammals and in particular not needed to provide therapeutic amounts of dsiRNA to human subjects. The advantages of the current invention include practical, uncomplicated methods of administration and therapeutic applications.

### SUMMARY

The invention relates to polymer compositions and methods for delivery of an iRNA agent, (e.g., an iRNA agent or siRNA agent) or other nucleic acid. In some embodiments, the nucleic acids which may be used in the polymer compositions and methods of the invention include iRNAs, siRNAs, single-stranded iRNAs, antagomirs, aptamers, antisense nucleic acids, decoy oligonucleotides, microRNAs (miRNAs), miRNA mimics, antimir, activating RNAs (RNAa), ribozymes, supermirs, U1 adaptor and the like. Derivatives of these nucleic acids may also be used.

Accordingly, in one aspect, the invention features a polymer composition of formula (I):

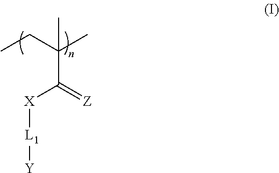

wherein
   Y is a nucleic acid or a ligand;
   L₁ is a straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R′)C(O)O, OC(O)NR′, CH(Q), phosphorus containing linkage, aryl, heteroaryl, heterocyclic, or cycloalkyl, where R′ is hydrogen, acyl, aliphatic or substituted aliphatic; Q is selected from OR₁₀, COR₁₀, CO₂R₁₀,

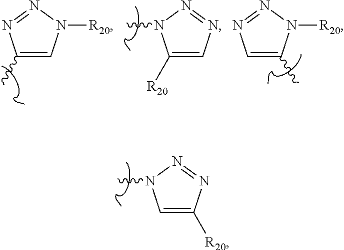

NR₂₀R₃₀, CONR₂₀R₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic, where R₂₀, R₃₀, R₄₀ and R₅₀ for each occurrence are independently selected from is hydrogen, acyl, aliphatic or substituted aliphatic, aryl, heteroaryl, heterocyclic, OR₁₀, COR₁₀, CO₂R₁₀, NR₁₀R₁₀′, R₂₀ and R₃₀ can be taken together to form a heterocyclic ring; R₁₀ and R₁₀′ are independently hydrogen, aliphatic, substituted aliphatic, aryl, heteroaryl, or heterocyclic;
X is absent, O, N(R′),
Z is O, S or NR′;
n is an integer between 5 to 20,000;
provided that at least one Y is a nucleic acid and Y further comprising at least two different ligands.

The details of one or more embodiments of the invention are set forth in the accompanying drawings and the description below. Other features, objects, and advantages of the invention will be apparent from this description, and from the claims. A person of ordinary skill in the art will readily recognize that additional embodiments of the invention exist. This application incorporates all cited references, patents, and patent applications by reference in their entirety.

### DETAILED DESCRIPTION

Accordingly, in one aspect, the invention features a polymer composition of formula (I):

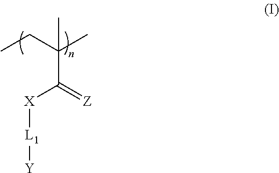

wherein
   Y is a nucleic acid or a ligand;
   L₁ is a straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R′)C(O)O, OC(O)NR′, CH(Q), phosphorus containing linkage, aryl, heteroaryl, heterocyclic, or cycloalkyl, where R′ is hydrogen, acyl, aliphatic or substituted aliphatic; Q is selected from OR₁₀, COR₁₀, CO₂R₁₀,

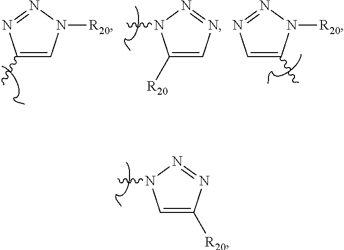

NR₂₀R₃₀, CONR₂₀R₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic, where R₂₀, R₃₀, R₄₀ and R₅₀ for each occurrence are independently selected from is hydrogen, acyl, aliphatic or substituted aliphatic, aryl, heteroaryl, heterocyclic, OR₁₀, COR₁₀, CO₂R₁₀, NR₁₀R₁₀′; R₂₀ and R₃₀ can be taken together to form a heterocyclic ring; R₁₀ and R₁₀′ are independently hydrogen, aliphatic, substituted aliphatic, aryl, heteroaryl, or heterocyclic;
X is absent, O, N(R′),
Z is O, S or NR′;
n is an integer between 5 to 20,000;
provided that at least one Y is a nucleic acid and at least two Y comprising two different ligands.

Accordingly, in one aspect, the invention features a polymer composition of formula (II):

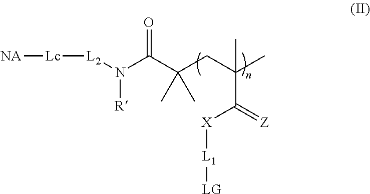

wherein
   NA is a nucleic acid;
   Lc is a cleavable linker;
   L₁ and L₂ are independently straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R)C(O)O, OC(O)NR′, CH(Q), phosphorus containing linkage, aryl, heteroaryl, heterocyclic, or cycloalkyl, where R′ is hydrogen, acyl, aliphatic or substituted aliphatic; Q is selected from OR₁₀, COR₁₀, CO₂R₁₀,

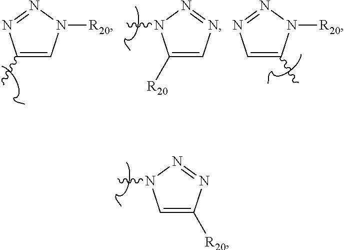

NR₂₀R₃₀, CONR₂₀R₃₀, CON(H)NR₂OR₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic, where R₂₀, R₃₀, R₄₀ and R₅₀ for each occurrence are independently selected from is hydrogen, acyl, aliphatic or substituted aliphatic, aryl, heteroaryl, heterocyclic, OR₁₀, COR₁₀, CO₂R₁₀, NR₁₀R₁₀′;
R₂₀ and R₃₀ can be taken together to form a heterocyclic ring; R₁₀ and R₁₀′ are independently hydrogen, aliphatic, substituted aliphatic, aryl, heteroaryl, or heterocyclic;
   X is absent, O, N(R′), Z is O, S or NR′;
   n is an integer between 5 to 20,000;
   LG is a ligand;
   and provided that there is a least two different LG groups.

Accordingly, in one aspect, the invention features a polymer composition of formula (III):

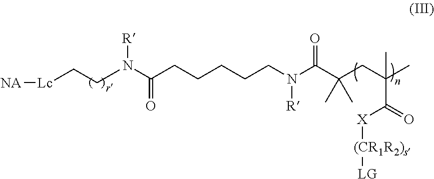

wherein
   NA is a nucleic acid;
   Lc is a cleavable linker;
   X is absent, O, N(R′);
   n is an integer between 5 to 20,000; s′ is 1-20;
   r′ is 1-10;
   R′ is independently for each occurrence hydrogen, acyl, aliphatic or substituted aliphatic;
   and LG is a ligand.

Accordingly, in one aspect, the invention features a polymer composition of formula (IV):

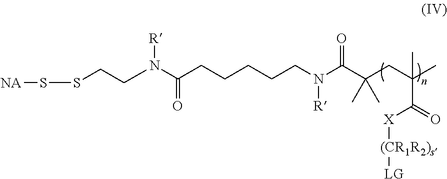

wherein
   NA is a nucleic acid;
   X is absent, O, N(R′);
   R′ is independently for each occurrence hydrogen, acyl, aliphatic or substituted aliphatic;
   n is an integer between 5 to 20,000;
   s′ is 1-20;
   and LG is a ligand.

Accordingly, in one aspect, the invention features a polymer composition of formula (V):

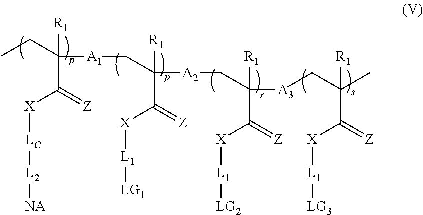

wherein
NA is a nucleic acid;
each of R₁ is independently hydrogen or C1-C6 alkyl;
A₁, A₂ and A₃ are either absent or a cleavable linker; preferably A₁, A₂ and A₃ are ester, disulfide, acetal, ketal, hydrazone.
p, q, r, and s are each independently an integer between 1 to 15,000;
Lc is a cleavable linker;
L₁ and L₂ are independently for each occurrence straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R)C(O)O, OC(O)NR′, CH(O), phosphorus containing linkage, aryl, heteroaryl, heterocyclic, or cycloalkyl, where R′ is hydrogen, acyl, aliphatic or substituted aliphatic; Q is selected from OR₁₀, COR₁₀, CO₂R₁₀,

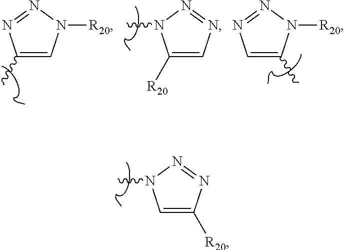

NR₂₀R₃₀, CONR₂₀R₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic, where R₂₀, R₃₀, R₄₀ and R₅₀ for each occurrence are independently selected from is hydrogen, acyl, aliphatic or substituted aliphatic, aryl, heteroaryl, heterocyclic, OR₁₀, COR₁₀, CO₂R₁₀, NR₁₀R₁₀′;
R₂₀ and R₃₀ can be taken together to form a heterocyclic ring; R₁₀ and R₁₀′ are independently hydrogen, aliphatic, substituted aliphatic, aryl, heteroaryl, or heterocyclic;
and LG₁, LG₂ and LG₃ are each independently selected from endosomolytic ligand, a targeting ligand, and PK modulator ligand.

Accordingly, in one aspect, the invention features a polymer composition of formula (VI):

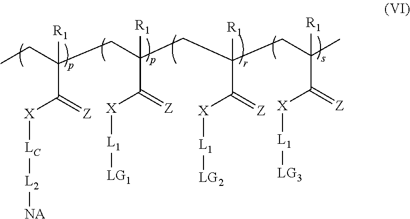

wherein
NA is a nucleic acid;
each of R₁ is independently hydrogen or C1-C6 alkyl;
p, q, r, and s are each independently an integer between 1 to 15,000;
Lc is a cleavable linker;
L₁ and L₂ are independently for each occurrence straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R′)C(O)O, OC(O)NR′, CH(Q), phosphorus containing linkage, aryl, heteroaryl, heterocyclic, or cycloalkyl, where R′ is hydrogen, acyl, aliphatic or substituted aliphatic; Q is selected from OR₁₀, COR₁₀, CO₂R₁₀,

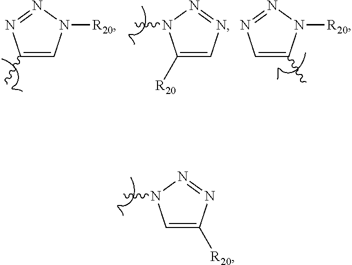

NR₂₀R₃₀, CONR₂₀R₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic, where R₂₀, R₃₀, R₄₀ and R₅₀ for each occurrence are independently selected from is hydrogen, acyl, aliphatic or substituted aliphatic, aryl, heteroaryl, heterocyclic, OR₁₀, COR₁₀, CO₂R₁₀, NR₁₀R₁₀′;
R₂₀ and R₃₀ can be taken together to form a heterocyclic ring; R₁₀ and R₁₀′ are independently hydrogen, aliphatic, substituted aliphatic, aryl, heteroaryl, or heterocyclic;
and LG₁, LG₂ and LG₃ are each independently selected from endosomolytic ligand, a targeting ligand, and PK modulator ligand.

Accordingly, in one aspect, the invention features a polymer composition of formula (VI):

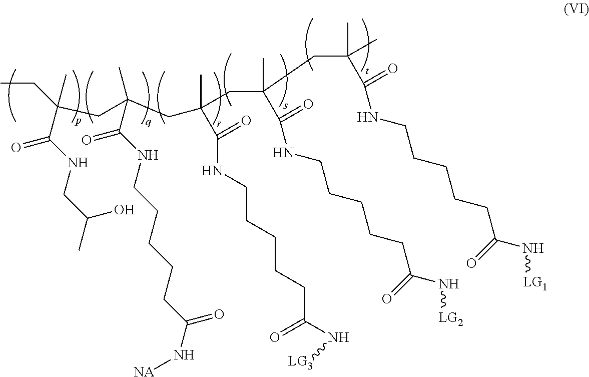

wherein NA is a nucleic acid; p, q, r, s and t are each independently an integer between 1 to 15,000; LG1, LG2 and LG3 are each independently selected from endosomolytic ligand, a targeting ligand, charge masking ligand, and PK modulator ligand.

In one embodiment, L₁ and L₂ are independently for each occurrence selected from the group consisting of

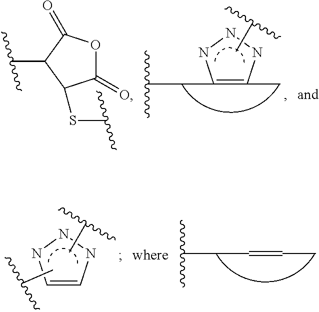

is a 5-10 membered ring.

In one embodiment, the copolymers of the invention comprises random copolymer, block copolymer, and amphiphilic copolymer.

In one embodiment, the multifunctional copolymers of the invention, are prepared from the monomers selected from the group consisting of:

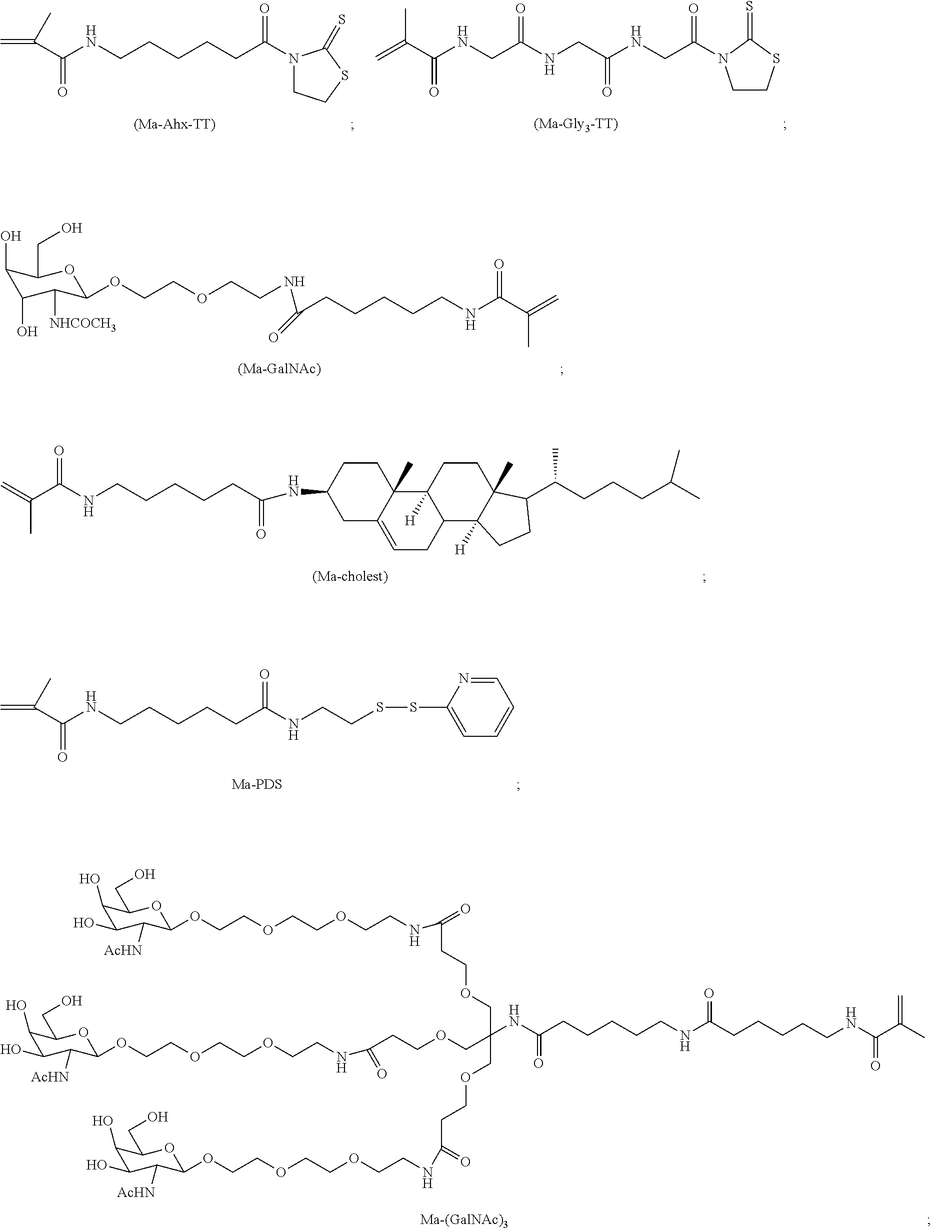

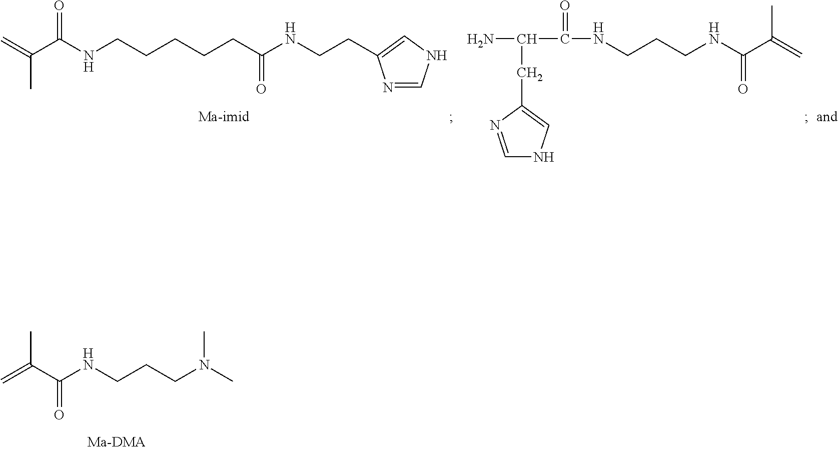 [SMILES: C=C(C)C(=O)NCCCCCC(=O)NCCc1c[nH]cn1 ; C=C(C)C(=O)NCCCNC(=O)C(N)Cc1c[nH]cn1 ; C=C(C)C(=O)NCCCN(C)C]

In one example, the multifunctional copolymer of the invention comprises various combinations of the following features:

| | Targeting/cell | |
|---|---|---|
| Scaffold | uptake/PK | endosomolytic |
| 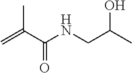 | 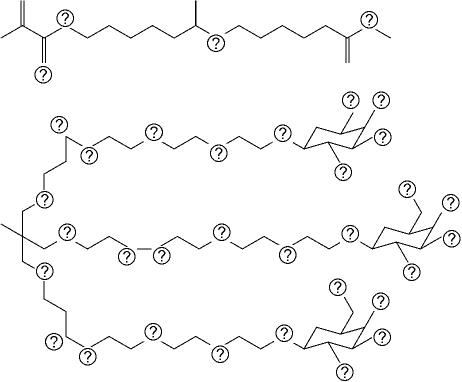 | 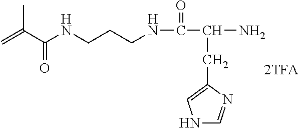 [SMILES: C=C(C)C(=O)NCCCNC(=O)C(N)Cc1c[nH]cn1] |
| 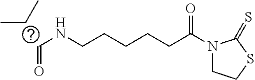 | 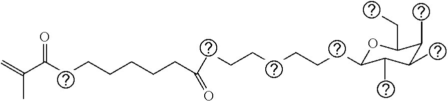 [SMILES: C=C(C)C(=O)CCCCCCC(=O)CCCCCCCC1OC(CC)C(C)C(C)C1C] | 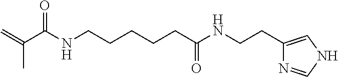 [SMILES: C=C(C)C(=O)NCCCCCC(=O)NCCc1c[nH]cn1] |
| 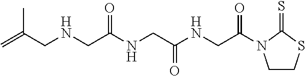 [SMILES: C=C(C)CNCC(=O)NCC(=O)NCC(=O)N1CCSC1=S] | 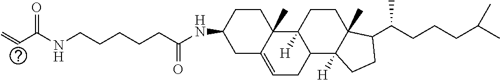 | 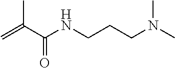 |
| 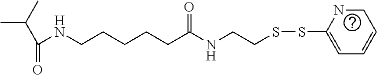 [SMILES: CC(C)C(=O)NCCCCCC(=O)NCCSSC1=CC=CCN1] | | |

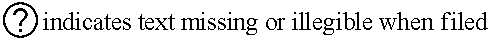

posomal formulations, the use of fusogenic lipids in the formulation has been the most common approach (Singh, R. S., Goncalves, C. et al. (2004). On the Gene Delivery Efficacies of pH-Sensitive Cationic Lipids via Endosomal Protonation. A Chemical Biology Investigation. Chem. Biol. 11, 713-723.). Other components, which exhibit pH-sensitiv

### Endosomolytic Ligands

For macromolecular drugs and hydrophilic drug molecules, which cannot easily cross bilayer membranes, entrapment in endosomal/lysosomal compartments of the cell is thought to be the biggest hurdle for effective delivery to their site of action. In recent years, a number of approaches and strategies have been devised to address this problem. For li e endosomolytic activity through protonation and/or pH-induced conformational changes, include charged polymers and peptides. Examples may be found in Hoffman, A. S., Stayton, P. S. et al. (2002). Design of “smart” polymers that can direct intracellular drug delivery. Polymers Adv. Technol. 13, 992-999; Kakudo, Chaki, T., S. et al. (2004). Transferrin-Modified Liposomes Equipped with a pH-Sensitive Fusogenic Peptide: An Artificial Viral-like Delivery System. Biochemistry 436, 5618-5628; Yessine, M. A. and Leroux, J. C. (2004). Membrane-destabilizing polyanions: interaction with lipid bilayers and endosomal escape of biomacromolecules. Adv. Drug Deliv. Rev. 56, 999-1021; Oliveira, S., van Rooy, I. et al. (2007). Fusogenic peptides enhance endosomal escape improving siRNA-induced silencing of oncogenes. Int. J. Pharm. 331, 211-4. They have generally been used in the context of drug delivery systems, such as liposomes or lipoplexes. For folate receptor-mediated delivery using liposomal formulations, for instance, a pH-sensitive fusogenic peptide has been incorporated into the liposomes and shown to enhance the activity through improving the unloading of drug during the uptake process (Turk, M. J., Reddy, J. A. et al. (2002). Characterization of a novel pH-sensitive peptide that enhances drug release from folate-targeted liposomes at endosomal pHs. Biochim. Biophys. Acta 1559, 56-68).

In certain embodiments, the endosomolytic ligands of the present invention may be polyanionic peptides or peptidomimetics which show pH-dependent membrane activity and/or fusogenicity. A peptidomimetic may be a small protein-like chain designed to mimic a peptide. A peptidomimetic may arise from modification of an existing peptide in order to alter the molecule's properties, or the synthesis of a peptide-like molecule using unnatural amino acids or their analogs. In certain embodiments, they have improved stability and/or biological activity when compared to a peptide. In certain embodiments, the endosomolytic ligand assumes its active conformation at endosomal pH (e.g., pH 5-6). The “active” conformation is that conformation in which the endosomolytic ligand promotes lysis of the endosome and/or transport of the modular composition of the invention, or its any of its components (e.g., a nucleic acid), from the endosome to the cytoplasm of the cell.

Libraries of compounds may be screened for their differential membrane activity at endosomal pH versus neutral pH using a hemolysis assay. Promising candidates isolated by this method may be used as components of the modular compositions of the invention. A method for identifying an endosomolytic ligand for use in the compositions and methods of the present invention may comprise: providing a library of compounds; contacting blood cells with the members of the library, wherein the pH of the medium in which the contact occurs is controlled; determining whether the compounds induce differential lysis of blood cells at a low pH (e.g., about pH 5-6) versus neutral pH (e.g., about pH 7-8).

Exemplary endosomolytic ligands include the GALA peptide (Subbarao et al., Biochemistry, 1987, 26: 2964-2972), the EALA peptide (Vogel et al., J. Am. Chem. Soc., 1996, 118: 1581-1586), and their derivatives (Turk et al., Biochem. Biophys. Acta, 2002, 1559: 56-68). In certain embodiments, the endosomolytic ligand may contain a chemical group (e.g., an amino acid) which will undergo a change in charge or protonation in response to a change in pH. The endosomolytic ligand may be linear or branched. Exemplary primary sequences of endosomolytic ligands include H₂N-(AALEALAEALEALAEALEALAEAAAAGGC)-CO₂H; H₂N-(AALAEALAEALAEALAEALAEALAAAAGGC)-CO₂H; and H₂N-(ALEALAEALEALAEA)-CONH₂.

Further examples of endosomolytic ligands include those in Table 1:

TABLE 1
Exemplary Endosomolytic ligands

| Name | Sequence (N to C) | Ref. |
|---|---|---|
| GALA | AALEALAEALEALAEALEALAEAAAAGGC | |
| EALA | AALAEALAEALAEALAEALAEALAAAAGGC | |
| | ALEALAEALEALAEA | |
| INF-7 | GLFEAIEGFIENGWEGMIWDYG | |
| Inf HA-2 | GLFGAIAGFIENGWEGMIDGWYG | |
| diINF-7 | GLF EAI EGFI ENGW EGMI DGWYGC | |
| | GLF EAI EGFI ENGW EGMI DGWYGC | |
| diINF3 | GLF EAI EGFI ENGW EGMI DGGC | |
| | GLF EAI EGFI ENGW EGMI DGGC | |
| GLF | GLFGALAEALAEALAEHLAEALAEALEALAAGGSC | |
| GALA-INF3 | GLFEAIEGFIENGWEGLAEALAEALEALAAGGSC | |
| INF-5 | GLF EAI EGFI ENGW EGnI DG K | |
| | GLF EAI EGFI ENGW EGnI DG | |
| JTS-1 | GLFEALLELLESLWELLLEA | |
| ppTG1 | GLFKALLKLLKSLWKLLLKA | |
| ppTG20 | GLFRALLRLLRSLWRLLLRA | |
| KALA | WEAKLAKALAKALAKHLAKALAKALKACEA | |
| HA | GLFFEAIAEFIEGGWEGLIEGC | |
| Melittin | GIGAVLKVLTTGLPALISWIKRKRQQ | |
| Histidine | CHK₆HC | |
| rich | | |

n, norleucine

In some embodiments, endosomolytic ligands can include imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

The endosomolytic ligand of this invention is a cellular compartmental release component, and may be any compound capable of releasing from any of the cellular compartments known in the art, such as the endosome, lysosome, endoplasmic reticulum (ER), golgi apparatus, microtubule, peroxisome, or other vesicular bodies with the cell.

In some embodiments, the membrane active functionality of the endosomolytic agent is masked when said endosomolytic agent is conjugated with the oligonucleotide. When the oligonucleotide reaches the endosome, the membrane active functionality is unmasked and the agent becomes active. The unmasking may be carried out more readily under the conditions found in the endosome than outside the endosome. For example, the membrane active functionality can be masked with a molecule through a cleavable linker that under goes cleavage in the endosome. Without wishing to be bound by theory, it is envisioned that upon entry into the endosome, such a linkage will be cleaved and the masking agent released from the endosomolytic agent.

In some embodiments, the masking agent has a cleavable linker that upon cleavage release a functional group that can cleave the linkage between the masking agent and the active functional group of the endosomolytic agent. One example is a masking agent linked to the endosomolytic agent through a amide type linkage, and having a S—S bond. Upon entry into the endosome, the S—S bond can be cleaved releasing free thiols that can then cleave the amide linkage between the masking agent and the endosomolytic agents either inter or intra molecularly. United States Patent Application Publication No. 2008/0281041 describes some masked endosomolytic polymers that are amenable to the present invention.

Lipids having membrane activity are also amenable to the present invention as endosomolytic agents. Such lipids are also described as fusogenic lipids. These fusogenic lipids are thought to fuse with and consequently destabilize a membrane. Fusogenic lipids usually have small head groups and unsaturated acyl chains. Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC).

The histidine-rich peptide H5WYG is a derivative of the N-terminal sequence of the HA-2 subunit of the influenza virus hemagglutinin in which 5 of the amino acids have been replaced with histidine residues. H5WYG is able to selectively destabilize membranes at a slightly acidic pH as the histidine residues are protonated.

In some embodiments, the endosomolytic ligand is a cell-permeation agent, preferably a helical cell-permeation agent. Preferably, the agent is amphipathic. The helical agent is preferably an alpha-helical agent, which preferably has a lipophilic and a lipophobic phase. A cell-permeation agent can be, for example, a cell permeation peptide, cationic peptide, amphipathic peptide or hydrophobic peptide, e.g. consisting primarily of Tyr, Trp and Phe, dendrimer peptide, constrained peptide or crosslinked peptide. In some embodiments, the cell permeation peptide can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP) containing a hydrophobic MTS can also be a targeting ligand. The cell permeation peptide can be a “delivery” peptide, which can carry large polar molecules including peptides, oligonucleotides, and protein across cell membranes. Some exemplary cell-permeation peptides are shown in Table 2.

TABLE 2
Exemplary Cell Permeation Peptides.

| Cell Permeation Peptide | Amino acid Sequence | Reference |
|---|---|---|
| Penetration | RQIKIWFQNRRMKWKK | Derossi et al., J. Biol. |
| | | Chem. 269: 10444, 1994 |
| Tat fragment | GRKKRRQRRRPPQC | Vives et al., J. Biol. |
| (48-60) | | Chem., 272: 16010, 1997 |
| Signal Sequence-based | GALFLGWLGAAGSTMGAWSQPKKKRKV | Chaloin et al., Biochem. |
| peptide | | Biophys. Res. Commun., |
| | | 243: 601, 1998 |
| PVEC | LLIILRRRIRKQAHAHSK | Elmquist et al., Exp. Cell |
| | | Res., 269: 237, 2001 |
| Transportan | GWTLNSAGYLLKINLKALAALAKKIL | Pooga et al., FASEB J., |
| | | 12: 67, 1998 |
| Amphiphilic model peptide | KLALKLALKALKAALKLA | Oehlke et al., Mol. Ther., |
| | | 2: 339, 2000 |
| Arg₉ | RRRRRRRRR | Mithchell et al., J. Pept. |
| | | Res., 56: 318, 2000 |
| Bacterial cell wall permeating | KFFKFFKFFK | |
| LL-37 | LLGDFFRKSKEKIGKEFKRIVQRIKDF | |
| | LRNLVPRTES | |
| Cecropin P1 | SWLSKTAKKLENSAKKRISEGIAIAIQGGPR | |
| α-defensin | ACYCRIPACIAGERRYGTCIYQGRLWAFCC | |
| b-defensin | DHYNCVSSGGQCLYSACPIFTKIQGTCYRGK | |
| | AKCCK | |
| Bactenecin | RKCRIVVIRVCR | |
| PR-3 | RRRPRPPYLPRPRPPPFFPPRLPPRIPPGFP | |
| | PRFPPRFPGKR-NH2 | |
| Indolicidin | ILPWKWPWWPWRR-NH2 | |

Cell-permeation peptides can be linear or cyclic, and include D-amino acids, non-peptide or pseudo-peptide linkages, peptidyl mimics. In addition the peptide and peptide mimics can be modified, e.g. glycosylated or methylated. Synthetic mimics of targeting peptides are also included.

In certain embodiments, more than one endosomolytic ligand may be incorporated in the modular composition of the invention. In some embodiments, this will entail incorporating more than one of the same endosomolytic ligand into the modular composition. In other embodiments, this will entail incorporating two or more different endosomolytic ligands into the modular composition.

These endosomolytic ligands may mediate endosomal escape by, for example, changing conformation at endosomal pH. In certain embodiments, the endosomolytic ligands may exist in a random coil conformation at neutral pH and rearrange to an amphipathic helix at endosomal pH. As a consequence of this conformational transition, these peptides may insert into the lipid membrane of the endosome, causing leakage of the endosomal contents into the cytoplasm. Because the conformational transition is pH-dependent, the endosomolytic ligands can display little or no fusogenic activity while circulating in the blood (pH ˜7.4). Fusogenic activity is defined as that activity which results in disruption of a lipid membrane by the endosomolytic ligand. One example of fusogenic activity is the disruption of the endosomal membrane by the endosomolytic ligand, leading to endosomal lysis or leakage and transport of one or more components of the modular composition of the invention (e.g., the nucleic acid) from the endosome into the cytoplasm.

In addition to the hemolysis assay described herein, suitable endosomolytic ligands can be tested and identified by a skilled artisan using other methods. For example, the ability of a compound to respond to, e.g., change charge depending on, the pH environment can be tested by routine methods, e.g., in a cellular assay. In certain embodiments, a test compound is combined with or contacted with a cell, and the cell is allowed to internalize the test compound, e.g., by endocytosis. An endosome preparation can then be made from the contacted cells and the endosome preparation compared to an endosome preparation from control cells. A change, e.g., a decrease, in the endosome fraction from the contacted cell vs. the control cell indicates that the test compound can function as a fusogenic agent. Alternatively, the contacted cell and control cell can be evaluated, e.g., by microscopy, e.g., by light or electron microscopy, to determine a difference in the endosome population in the cells. The test compound and/or the endosomes can labeled, e.g., to quantify endosomal leakage.

In another type of assay, a modular composition described herein is constructed using one or more test or putative fusogenic agents. The modular composition can be constructed using a labeled nucleic acid. The ability of the endosomolytic ligand to promote endosomal escape, once the modular composition is taken up by the cell, can be evaluated, e.g., by preparation of an endosome preparation, or by microscopy techniques, which enable visualization of the labeled nucleic acid in the cytoplasm of the cell. In certain other embodiments, the inhibition of gene expression, or any other physiological parameter, may be used as a surrogate marker for endosomal escape.

In other embodiments, circular dichroism spectroscopy can be used to identify compounds that exhibit a pH-dependent structural transition.

A two-step assay can also be performed, wherein a first assay evaluates the ability of a test compound alone to respond to changes in pH, and a second assay evaluates the ability of a modular composition that includes the test compound to respond to changes in pH.

### Targeting Ligands

The modular compositions of the present invention comprise a targeting ligand. In some embodiments, this targeting ligand may direct the modular composition to a particular cell. For example, the targeting ligand may specifically or non-specifically bind with a molecule on the surface of a target cell. The targeting moiety can be a molecule with a specific affinity for a target cell. Targeting moieties can include antibodies directed against a protein found on the surface of a target cell, or the ligand or a receptor-binding portion of a ligand for a molecule found on the surface of a target cell. For example, the targeting moiety can recognize a cancer-specific antigen (e.g., CA15-3, CA19-9, CEA, or HER2/neu) or a viral antigen, thus delivering the iRNA to a cancer cell or a virus-infected cell. Exemplary targeting moieties include antibodies (such as IgM, IgG, IgA, IgD, and the like, or a functional portions thereof), ligands for cell surface receptors (e.g., ectodomains thereof).

Table 3 provides examples of a number of antigens which can be used to target selected cells.

TABLE 3
Exemplary antigens for targeting specific cells

| ANTIGEN | Exemplary tumor tissue |
|---|---|
| CEA (carcinoembryonic antigen) | colon, breast, lung |
| PSA (prostate specific antigen) | prostate cancer |
| CA-125 | ovarian cancer |
| CA 15-3 | breast cancer |
| CA 19-9 | breast cancer |
| HER2/neu | breast cancer |
| α-feto protein | testicular cancer, hepatic |
| | cancer |
| β-HCG (human chorionic gonadotropin) | testicular cancer, |
| | choriocarcinoma |
| MUC-1 | breast cancer |
| Estrogen receptor | breast cancer, uterine cancer |
| Progesterone receptor | breast cancer, uterine cancer |
| EGFr (epidermal growth factor receptor) | bladder cancer |

Ligand-mediated targeting to specific tissues through binding to their respective receptors on the cell surface offers an attractive approach to improve the tissue-specific delivery of drugs. Specific targeting to disease-relevant cell types and tissues may help to lower the effective dose, reduce side effects and consequently maximize the therapeutic index. Carbohydrates and carbohydrate clusters with multiple carbohydrate motifs represent an important class of targeting ligands, which allow the targeting of drugs to a wide variety of tissues and cell types. For examples, see Hashida, M., Nishikawa, M. et al. (2001) Cell-specific delivery of genes with glycosylated carriers. Adv. Drug Deliv. Rev. 52, 187-9; Monsigny, M., Roche, A.-C. et al. (1994). Glycoconjugates as carriers for specific delivery of therapeutic drugs and genes. Adv. Drug Deliv. Rev. 14, 1-24; Gabius, S., Kayser, K. et al. (1996). Endogenous lectins and neoglycoconjugates. A sweet approach to tumor diagnosis and targeted drug delivery. Eur. J. Pharm. and Biopharm. 42, 250-261; Wadhwa, M. S., and Rice, K. G. (1995) Receptor mediated glycotargeting. J. Drug Target. 3, 111-127.

One of the best characterized receptor-ligand pairs is the asialoglycoprotein receptor (ASGP-R), which is highly expressed on hepatocytes and which has a high affinity for D-galactose as well as N-acetyl-D-galactose (GalNAc). Those carbohydrate ligands have been successfully used to target a wide variety of drugs and even liposomes or polymeric carrier systems to the liver parenchyma. For examples, see Wu, G. Y., and Wu, C. H. (1987) Receptor-mediated in vitro gene transformation by a soluble DNA carrier system. J. Biol. Chem. 262, 4429-4432; Biessen, E. A. L., Vietsch, H., Rump, E. T., Flutter, K., Bijsterbosch, M. K., and Van Berkel, T. J. C. (2000) Targeted delivery of antisense oligonucleotides to parenchymal liver cells in vivo. Methods Enzymol. 313, 324-342; Zanta, M.-A., Boussif, O., Adib, A., and Behr, J.-P. (1997) In Vitro Gene Delivery to Hepatocytes with Galactosylated Polyethylenimine. Bioconjugate Chem. 8, 839-844; Managit, C., Kawakami, S. et al. (2003). Targeted and sustained drug delivery using PEGylated galactosylated liposomes. Int. J. Pharm. 266, 77-84; Sato, A., Takagi, M. et al. (2007). Small interfering RNA delivery to the liver by intravenous administration of galactosylated cationic liposomes in mice. Biomaterials 28; 1434-42.

The Mannose receptor, with its high affinity to D-mannose represents another important carbohydrate-based ligand-receptor pair. The mannose receptor is highly expressed on specific cell types such as macrophages and possibly dendritic cells Mannose conjugates as well as mannosylated drug carriers have been successfully used to target drug molecules to those cells. For examples, see Biessen, E. A. L., Noorman, F. et al. (1996). Lysine-based cluster mannosides that inhibit ligand binding to the human mannose receptor at nanomolar concentration. J. Biol. Chem. 271, 28024-28030; Kinzel, O., Fattori, D. et al. (2003). Synthesis of a functionalized high affinity mannose receptor ligand and its application in the construction of peptide-, polyamide- and PNA-conjugates. J. Peptide Sci. 9, 375-385; Barratt, G., Tenu, J. P. et al. (1986). Preparation and characterization of liposomes containing mannosylated phospholipids capable of targeting drugs to macrophages. Biochim. Biophys. Acta 862, 153-64; Diebold, S. S., Plank, C. et al. (2002). Mannose Receptor-Mediated Gene Delivery into Antigen Presenting Dendritic Cells. Somat. Cell Mol. Genetics. 27, 65-74.

Carbohydrate based targeting ligands include, but are not limited to, D-galactose, multivalent galactose, N-acetyl-D-galactose (GalNAc), multivalent GalNAc, e.g. GalNAC2 and GalNAc3; D-mannose, multivalent mannose, multivalent lactose, N-acetyl-galactosamine, N-acetyl-gulucosamine, multivalent fucose, glycosylated polyaminoacids and lectins. The term multivalent indicates that more than one monosaccharide unit is present. Such monosaccharide subunits may be linked to each other through glycosidic linkages or linked to a scaffold molecule.

Lipophilic moieties, such as cholesterol or fatty acids, when attached to highly hydrophilic molecules such as nucleic acids can substantially enhance plasma protein binding and consequently circulation half life. In addition, binding to certain plasma proteins, such as lipoproteins, has been shown to increase uptake in specific tissues expressing the corresponding lipoprotein receptors (e.g., LDL-receptor or the scavenger receptor SR-B1). For examples, see Bijsterbosch, M. K., Rump, E. T. et al. (2000). Modulation of plasma protein binding and in vivo liver cell uptake of phosphorothioate oligodeoxynucleotides by cholesterol conjugation. Nucleic Acids Res. 28, 2717-25; Wolfrum, C., Shi, S. et al. (2007). Mechanisms and optimization of in vivo delivery of lipophilic siRNAs. Nat. Biotechnol. 25, 1149-57. Lipophilic conjugates can therefore also be considered as a targeted delivery approach and their intracellular trafficking could potentially be further improved by the combination with endosomolytic agents.

Exemplary lipophilic moieties that enhance plasma protein binding include, but are not limited to, sterols, cholesterol, fatty acids, cholic acid, lithocholic acid, dialkylglycerides, diacylglyceride, phospholipids, sphingolipids, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, phenoxazine, aspirin, naproxen, ibuprofen, vitamin E and biotin etc.

Folates represent another class of ligands which has been widely used for targeted drug delivery via the folate receptor. This receptor is highly expressed on a wide variety of tumor cells, as well as other cells types, such as activated macrophages. For examples, see Matherly, L. H. and Goldman, I. D. (2003). Membrane transport of folates. Vitamins Hormones 66, 403-456; Sudimack, J. and Lee, R. J. (2000). Targeted drug delivery via the folate receptor. Adv. Drug Delivery Rev. 41, 147-162. Similar to carbohydrate-based ligands, folates have been shown to be capable of delivering a wide variety of drugs, including nucleic acids and even liposomal carriers. For examples, see Reddy, J. A., Dean, D. et al. (1999). Optimization of Folate-Conjugated Liposomal Vectors for Folate Receptor-Mediated Gene Therapy. J. Pharm. Sci. 88, 1112-1118; Lu, Y. and Low P. S. (2002). Folate-mediated delivery of macromolecular anticancer therapeutic agents. Adv. Drug Delivery Rev. 54, 675-693; Zhao, X. B. and Lee, R. J. (2004). Tumor-selective targeted delivery of genes and antisense oligodeoxyribonucleotides via the folate receptor; Leamon, C. P., Cooper, S. R. et al. (2003). Folate-Liposome-Mediated Antisense Oligodeoxynucleotide Targeting to Cancer Cells: Evaluation in Vitro and in Vivo. Bioconj. Chem. 14, 738-747.

U.S. patent application Ser. No. 12/328,537, filed Dec. 4, 2008 and Ser. No. 12/328,528, filed Dec. 4, 2008 describe a number of folate and carbohydrate targeting ligands that are amenable to the modular compositions of the present invention. Contents of these patent applications are herein incorporated by reference in their entirety.

The targeting ligands also include proteins, peptides and peptidomimmetics that can target cell markers, e.g. markers enriched in proliferating cells. A peptidomimetic (also referred to herein as an oligopeptidomimetic) is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long Such peptides include, but are not limited to, RGD containing peptides and peptidomimmetics that can target cancer cells, in particular cells that exhibit αᵥβ₃ (alpha.v.beta.3) integrin. Targeting peptides can be linear or cyclic, and include D-amino acids, non-peptide or pseudo-peptide linkages, peptidyl mimics. In addition the peptide and peptide mimics can be modified, e.g. glycosylated or methylated. Synthetic mimics of targeting peptides are also included.

The targeting ligands can also include other receptor binding ligands such as hormones and hormone receptor binding ligands. A targeting ligand can be a thyrotropin, melanotropin, lectin, glycoprotein, surfactant protein A, mucin, glycosylated polyaminoacids, transferrin, bisphosphonate, polyglutamate, polyaspartate, a lipid, folate, vitamin B12, biotin, or an aptamer. Table 4 shows some examples of targeting ligands and their associated receptors.

TABLE 4
Liver Targeting Ligands and their associated receptors

| Liver Cells | Ligand | Receptor |
|---|---|---|
| 1) Parenchymal Cell (PC) | Galactose | ASGP-R |
| (Hepatocytes) | | (Asiologlycoprotein receptor) |
| | Gal NAc | ASPG-R |
| | (n-acetyl-galactosamine) | Gal NAc Receptor |
| | Lactose | |
| | Asialofetuin | ASPG-r |
| 2) Sinusoidal Endothelial | Hyaluronan | Hyaluronan receptor |
| Cell (SEC) | Procollagen | Procollagen receptor |
| | Negatively charged molecules | Scavenger receptors |
| | Mannose | Mannose receptors |
| | N-acetyl Glucosamine | Scavenger receptors |
| | Immunoglobulins | Fc Receptor |
| | LPS | CD14 Receptor |
| | Insulin | Receptor mediated transcytosis |
| | Transferrin | Receptor mediated transcytosis |
| | Albumins | Non-specific |
| | Sugar-Albumin conjugates | |
| | Mannose-6-phosphate | Mannose-6-phosphate receptor |
| 3) Kupffer Cell (KC) | Mannose | Mannose receptors |
| | Fucose | Fucose receptors |
| | Albumins | Non-specific |
| | Mannose-albumin conjugates | |

When two or more targeting ligands are present, such targeting ligands may all be the same or different targeting ligands that target the same cell/tissue/organ.

In addition to the endosomolytic ligand and the targeting ligand, the modular composition may comprise one or more other moieties/ligands that may enhance circulation half life and/or cellular uptake. These can include naturally occurring substances, such as a protein (e.g., human serum albumin (HSA), low-density lipoprotein (LDL), high-density lipoprotein (HDL), or globulin); or a carbohydrate (e.g., a dextran, pullulan, chitin, chitosan, inulin, cyclodextrin or hyaluronic acid). These moieties may also be a recombinant or synthetic molecule, such as a synthetic polymer or synthetic polyamino acids. Examples include polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-5K, PEG-10K, PEG-12K, PEG-15K, PEG-20K, PEG-40K), methyl-PEG (mPEG), [mPEG]₂, polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Oligonucleotides and oligomeric compounds that comprise a number of phosphorothioate linkages are known in the art to bind to serum protein, thus short oligonucleotides, e.g. oligonucleotides of about 5 bases, 10 bases, 15 bases or 20 bases, and non-nucleosidic oligomeric compounds comprising multiple phosphorothioate linkages can be used to enhance the circulation half life of the modular composition of the invention. In addition, oligonucleotides, e.g. aptamers, that bind serum ligands (e.g. serum proteins) can also be used to enhance the circulation half life of the modular composition of the invention. These oligonucleotides and aptamers may comprise any nucleic acid modification, e.g. sugar modification, backbone modification or nucleobase modification, described in this application.

Ligands that increase the cellular uptake of the modular composition, may also be present in addition to the endosomolytic ligand and the targeting ligand. Exemplary ligands that enhance cellular uptake include vitamins. These are particularly useful for targeting cells/tissues/organs characterized by unwanted cell proliferation, e.g., of the malignant or non-malignant type, e.g., cancer cells. Exemplary vitamins include vitamin A, E, and K. Other exemplary vitamins include B vitamin, e.g., folic acid, B12, riboflavin, biotin, pyridoxal or other vitamins or nutrients taken up by cancer cells.

The ligand can be a substance, e.g, a drug, which can increase the uptake of the modular composition into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the modular composition into the cell by activating an inflammatory response, for example. Exemplary ligands that would have such an effect include tumor necrosis factor alpha (TNFalpha), interleukin-1 beta, or gamma interferon.

In some embodiments, such a ligand is a cell-permeation agent, preferably a helical cell-permeation agent. Preferably, the agent is amphipathic. The helical agent is preferably an alpha-helical agent, which preferably has a lipophilic and a lipophobic phase.

Other ligands that can be present in the modular composition of the invention include, dyes and reporter groups for monitoring distribution, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, mercapto, amino, polyamino, alkyl, substituted alkyl, radiolabeled markers, enzymes, haptens (e.g. biotin), synthetic ribonucleases (e.g., imidazole, bisimidazole, histamine, imidazole clusters, acridine-imidazole conjugates, Eu3+ complexes of tetraazamacrocycles, dinitrophenyl, HRP and AP.

In some embodiments, a single ligand may have more than one property, e.g. ligand has both endosomolytic and targeting properties.

### PK Modulators

PK modulator stands for pharmacokinetic modulator. PK modulator include lipophiles, bile acids, steroids, phospholipid analogues, peptides, protein binding agents, PEG, vitamins etc. Examplary PK modulator include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g. oligonucleotides of about 5 bases, 10 bases, 15 bases or 20 bases, comprising multiple of phosphorothioate linkages in the backbaone are also amenable to the present invention as ligands (e.g. as PK modulating ligands).

### Masking Agent

A masking agent comprises a molecule which, when linked to a polymer, shields, inhibits or inactivates one or more properties (biophysical or biochemical characteristics) of the polymer. A masking agent can also add an activity or function to the polymer that the polymer did not have in the absence of the asking agent. Properties of polymers that may be masked include: membrane activity, endosomo lytic activity, charge, effective charge, transfection activity, serum interaction, cell interaction, and toxicity. Masking agents can also inhibit or prevent aggregation of the polynucleotide-polymer conjugate in physiological conditions. Masking agents of the invention may be selected from the group consisting of: steric stabilizers, targeting groups, and charge modifiers. Multiple masking agents can be reversibly linked to a single polymer. To inactivate a property of a polymer, it may be necessary to link more than one masking agent to the polymer. A sufficient number of masking agents are linked to the polymer to achieve the desired level of inactivation. The desired level of modification of a polymer by attachment of masking agent(s) is readily determined using appropriate polymer activity assays. For example, if the polymer possesses membrane activity in a given assay, a sufficient level of masking agent is linked to the polymer to achieve the desired level of inhibition of membrane activity in that assay. A sufficient number of masking agent can be reversibly linked to the polymer to inhibit aggregation of the polymer in physiologically conditions. More than one species of masking agent may be used. For example, both steric stabilizers and targeting groups may be linked to a polymer. Steric stabilizers and targeting groups may or may not also function as charge modifiers. The masking agents of the invention are reversibly linked to the polymer. As used herein, a masking agent is reversibly linked to a polymer if reversal of the linkage results in restoration of the masked activity of the polymer: Masking agents are linked to the polymer through the formation of reversible covalent linkages with reactive groups on the polymer. Reactive groups may be selected from the groups comprising: amines, alcohols, thiols, hydrazides, aldehydes, carboxyls, etc. From one to all of the reactive groups or charged groups on a polymer may be reversibly modified. In one embodiment, at least two masking agents are reversibly linked to the polymer. In another embodiment, masking agents are reversibly linked to about 20%, 30%, 40%, 50%, 60%, 70%, or 80% of the reactive groups on the polymer. In another embodiment, masking agents are reversibly linked to about 20%, 30%, 40%, 50%, 60%, 70%, or 80% of the charged groups on the polymer. In another embodiment, the percentage of masking agents reversibly linked the polymer to charged groups on the polymer is about 20%, 30%, 40%, 50%, 60%, 70%, or 80%. As used herein, a polymer is masked if one or more properties of the polymer is inhibited or inactivated by attachment of one or more masking agents. A polymer is reversibly masked if cleavage of bonds linking the masking agents to the polymer results in restoration of the polymer's masked property.

In one embodiment, the amine masking agents of the invention are selected from:

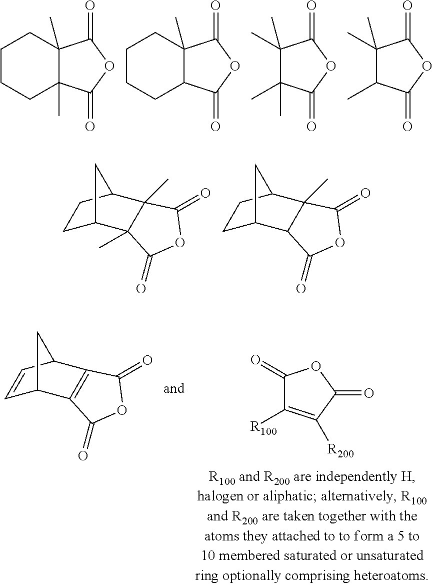

### Enhanced Permeability and Retention

In certain embodiments, the modular composition of the invention may be targeted to a site via the enhanced permeability and retention (EPR) effect. The EPR effect is the property by which certain sizes of molecules, typically macromolecules, tend to accumulate in, for example, tumor tissue to a greater extent than in normal tissue. Without being bound by theory, the general explanation for this phenomenon is that the blood vessels supplying a tumor are typically abnormal in their architecture, containing wide fenestrations which permit the diffusion of macromolecules from the blood. Moreover, tumors typically lack effective lymphatic drainage, leading to the accumulation of molecules that diffuse from the blood. A person of ordinary skill in the art will recognize that such methods of targeting may also be useful for other conditions in which abnormal vasculature enable access to a specific site, with or without compromised lymphatic drainage.

Representative United States patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; each of which is herein incorporated by reference.

### Linkers

In certain embodiments, the covalent linkages between any of the three components of the modular composition of the invention may be mediated by a linker. This linker may be cleavable or non-cleavable, depending on the application. In certain embodiments, a cleavable linker may be used to release the nucleic acid after transport from the endosome to the cytoplasm. The intended nature of the conjugation or coupling interaction, or the desired biological effect, will determine the choice of linker group.

Linker groups may be connected to the oligonucleotide strand(s) at a linker group attachment point (LAP) and may include any C₁-C₁₀₀ carbon-containing moiety, (e.g., C₁-C₇₅, C₁-C₅₀, C₁-C₂₀, C₁-C₁₀; C₁, C₂, C₃, C₄, C_{S}, C₆, C₇, C₈, C₉, or C₁₀), in some embodiments having at least one oxygen atom, at least one phosphorous atom, and/or at least one nitrogen atom. In some embodiments, the phosphorous atom forms part of a terminal phosphate, or phosphorothioate, group on the linker group, which may serve as a connection point for the nucleic acid strand. In certain embodiments, the nitrogen atom forms part of a terminal ether, ester, amino or amido (NHC(O)—) group on the linker group, which may serve as a connection point for the endosomolytic ligand or targeting ligand. Preferred linker groups (underlined) include LAP-X—(CH₂)ₙNH—; LAP-X—C(O)(CH₂)ₙNH—; LAP-X—NR″″(CH₂)ₙNH—, LAP-X—C(O)—(CH₂)ₙ—C(O)—; LAP-X—C(O)—(CH₂)ₙ—C(O)O—; LAP-X—C(O)—O—; LAP-X—C(O)—(CH₂)ₙ—NH—C(O)—; LAP-X—C(O)—(CH₂)ₙ—; LAP-X—C(O)—NH—; LAP-X—C(O)—; LAP-X—(CH₂)ₙ—C(O)—; LAP-X—(CH₂)ₙ—C(O)O—; LAP-X—(CH₂)ₙ—; or LAP-X—(CH₂)ₙ—NH—C(O)—; in which —X is (—O— (R″″O)P(O)—O)ₘ, (—O—R″″O)P(S)—O—)ₘ, (—O—(R″″S)P(O)—O)ₘ, (—O—(R″″S)P(S)—O)ₘ, (—O— (R″″O)P(O)—S)ₘ, (—S—(R″″O)P(O)—O)ₘ, or nothing, n is 1-20 (e.g., 1, 2, 3, 4, 5, 6, 7, 8, 9, 10, 11, 12, 13, 14, 15, 16, 17, 18, 19, or 20), m is 1 to 3, and R″″ is H or C₁-C₆ alkyl. Preferably, n is 5, 6, or 11. In other embodiments, the nitrogen may form part of a terminal oxyamino group, e.g., —ONH₂, or hydrazino group, —NHNH₂. The linker group may optionally be substituted, e.g., with hydroxy, alkoxy, perhaloalkyl, and/or optionally inserted with one or more additional heteroatoms, e.g., N, O, or S. Certain linker groups may include, e.g., LAP-X—(CH₂)ₙNH—; LAP-X—C(O)(CH₂)ₙNH—; LAP-X—NR″″(CH₂)ₙNH—; LAP-X—(CH₂)ₙONH—; LAP-X—C(O)(CH₂)ₙONH—; LAP-X—NR″″(CH₂)ₙONH—; LAP-X—(CH₂)ₙNHNH₂—, LAP-X—C(O)(CH₂)ₙNHNH₂—; LAP-X—NR″″(CH₂)ₙNHNH₂—; LAP-X—C(O)—(CH₂)ₙ—C(O)—; LAP-X—C(O)—(CH₂)ₙ—C(O)O—; LAP-X—C(O)—O—; LAP-X—C(O)—(CH₂)ₙ—NH—C(O)—; LAP-X—C(O)—(CH₂)ₙ—; LAP-X—C(O)—NH—; LAP-X—C(O)—; LAP-X—(CH₂)ₙ—C(O)—; LAP-X—(CH₂)ₙ—C(O)O—; LAP-X—(CH₂)ₙ—; or LAP-X—(CH₂)ₙ—NH—C(O)—. In some embodiments, amino terminated linker groups (e.g., NH₂, ONH₂, NH₂NH₂) can form an imino bond (i.e., C═N) with the ligand. In some embodiments, amino terminated linker groups (e.g., NH₂, ONH₂, NH₂NH₂) can be acylated, e.g., with C(O)CF₃.

In some embodiments, the linker group can terminate with a mercapto group (i.e., SH) or an olefin (e.g., CH═CH₂). For example, the linker group can be LAP-X—(CH₂)ₙ—SH, LAP-X—C(O)(CH₂)ₙSH, LAP-X—(CH₂)ₙ—(CH═CH₂), or LAP-X—C(O)(CH₂)CH═CH₂, in which X and n can be as described for the linker groups above. In certain embodiments, the olefin can be a Diels-Alder diene or dienophile. The linker group may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments the linker group may include an electrophilic moiety, preferably at the terminal position of the linker group. Certain electrophilic moieties include, e.g., an aldehyde, alkyl halide, mesylate, tosylate, nosylate, or brosylate, or an activated carboxylic acid ester, e.g., an NHS ester, or a pentafluorophenyl ester. Other linker groups (underlined) include LAP-X—(CH₂)ₙCHO; LAP-X—C(O)(CH₂)ₙCHO; or LAP-X—NR″″(CH₂)ₙCHO, in which n is 1-6 and R″″ is C₁-C₆ alkyl; or LAP-X—(CH₂)ₙC(O))NHS; LAP-X—C(O)(CH₂)ₙC(O)ONHS; or LAP-X—NR″″(CH₂)ₙC(O)ONHS, in which n is 1-6 and R″″ is C₁-C₆ alkyl; LAP-X—(CH₂)ₙC(O)OC₆F₅; LAP-X—C(O)(CH₂)ₙC(O)OC₆F₅; or LAP-X—NR″″(CH₂)ₙC(O)OC₆F₅, in which n is 1-11 and R″″ is C₁-C₆ alkyl; or —(CH₂)ₙCH₂LG; LAP-X—C(O)(CH₂)ₙCH₂LG; or LAP-X—NR″″(CH₂)ₙCH₂LG, in which X, R″″ and n can be as described for the linker groups above (LG can be a leaving group, e.g., halide, mesylate, tosylate, nosylate, brosylate). In some embodiments, coupling the -linker group to the endosomolytic ligand or targeting ligand can be carried out by coupling a nucleophilic group of the endosomolytic ligand or targeting ligand with an electrophilic group on the linker group.

In other embodiments, other protected amino groups can be at the terminal position of the linker group, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

In any of the above linker groups, in addition, one, more than one, or all, of the n-CH₂— groups may be replaced by one or a combination of, e.g., X, as defined above, —Y—(CH₂)ₘ—, —Y—(C(CH₃)H)ₘ—, —Y—C((CH₂)ₚCH₃)H)ₘ—, —Y—(CH₂—C(CH₃)H)ₘ—, —Y—(CH₂—C((CH₂)ₚCH₃)H)ₘ—, —CH═CH—, or —C≡C—, wherein Y is O, S, Se, S—S, S(O), S(O)₂, m is 1-4 and p is 0-4.

Where more than one endosomolytic ligand or targeting ligand is present on the same modular composition, the more than one endosomolytic ligand or targeting ligand may be linked to the oligonucleotide strand or an endosomolytic ligand or targeting ligand in a linear fashion, or by a branched linker group.

In some embodiments, the linker group is a branched linker group, and more in ceratin cases a symmetric branched linker group. The branch point may be an at least trivalent, but may be a tetravalent, pentavalent, or hexavalent atom, or a group presenting such multiple valencies. In some embodiments, the branch point is a glycerol, or glycerol triphosphate, group.

In some embodiments, the branchpoint is, —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiments, the branchpoint is a glycerol derivative.

In one embodiment, the linker is —[(P-Q-R)_{q}—X—(P′-Q′-R′)_{q′}]_{q″}-T-, wherein:

P, R, T, P′ and R′ are each independently for each occurrence absent, CO, NH, O, S, OC(O), NHC(O), CH₂, CH₂NH, CH₂O; NHCH(R^{a})C(O), —C(O)—CH(R^{a})—NH—, C(O)—(optionally substituted alkyl)-NH—, CH═N—O,

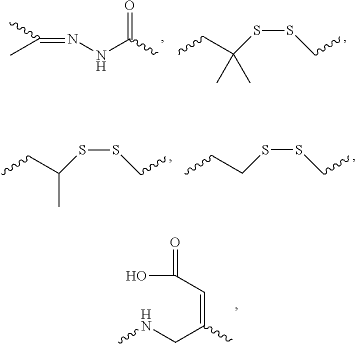

cyclyl, heterocycyclyl, aryl or heteroaryl;

Q and Q′ are each independently for each occurrence absent, —(CH₂)ₙ—, —C(R¹⁰⁰)(R²⁰⁰)(CH₂)ₙ—, —(CH₂)ₙC(R¹⁰⁰)(R²⁰⁰)—, —(CH₂CH₂O)ₘCH₂CH₂—, —(CH₂CH₂O)ₘCH₂CH₂NH—, aryl, heteroaryl, cyclyl, or heterocyclyl;

X is absent or a cleavable linker;

R^{a} is H or an amino acid side chain;

R¹⁰⁰ and R²⁰⁰ are each independently for each occurrence H, CH₃, OH, SH or N(R^{X})₂;

R^{X} is independently for each occurrence H, methyl, ethyl, propyl, isopropyl, butyl or benzyl;

q, q′ and q″ are each independently for each occurrence 0-30 and wherein the repeating unit can be the same or different;

n is independently for each occurrence 1-20; and

m is independently for each occurrence 0-50.

In some embodiments, a carrier monomer is also considered a linker. In those instances the term linker comprises the carrier monomer and the linker between the monomer and the ligand, e.g. endosomolytic ligand and targeting ligand.

In some embodiments, the linker comprises at least one cleavable linker.

### Cleavable Linker

A cleavable linker is one which is sufficiently stable outside the cell, but which upon entry into a target cell is cleaved to release the two parts the linker is holding together. In a preferred embodiment, the cleavable linker is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linker that is cleaved at a preferred pH, thereby releasing the cationic lipid from the ligand inside the cell, or into the desired compartment of the cell.

A linker can include a cleavable linker that is cleavable by a particular enzyme. The type of cleavable linker incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linker can be evaluated by testing the ability of a degradative agent (or condition) to cleave the candidate linking group. It will also be desirable to also test the candidate cleavable linker for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linkers

One class of cleavable linkers are redox cleavable linkers that are cleaved upon reduction or oxidation. An example of reductively cleavable linker is a disulphide linking group (—S—S—). To determine if a candidate cleavable linker is a suitable “reductively cleavable linker,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linkers

Phosphate-based cleavable linkers are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linkers

Acid cleavable linkers are linking groups that are cleaved under acidic conditions. In preferred embodiments acid cleavable linkers are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linkers. Examples of acid cleavable linkers include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Cleavable Linkers

Ester-based cleavable linkers are cleaved by enzymes such as esterases and amidases in cells. Examples of ester-based cleavable linkers include but are not limited to esters of alkylene, alkenylene and alkynylene groups. Ester cleavable linkers have the general formula —C(O)O—, or —OC(O)—. These candidates can be evaluated using methods analogous to those described above.

### Peptide-Based Cleaving Linking Groups

Peptide-based cleavable linkers are cleaved by enzymes such as peptidases and proteases in cells. Peptide-based cleavable linkers are peptide bonds formed between amino acids to yield oligopeptides (e.g., dipeptides, tripeptides etc.) and polypeptides. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linkers have the general formula —NHCHR^{A}C(O)NHCHR^{B}C(O)—, where R^{A} and R^{B} are the R groups of the two adjacent amino acids. These candidates can be evaluated using methods analogous to those described above.

Where more than one endosomolytic ligand or targeting ligand is present on the same modular composition, the more than one endosomolytic ligand or targeting ligand may be linked to the oligonucleotide strand or an endosomolytic ligand or targeting ligand in a linear fashion, or by a branched linker group.

iRNA Agents

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an RNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The mismatches, particularly in the antisense strand, are most tolerated in the terminal regions and if present may be in a terminal region or regions, e.g., within 6, 5, 4, or 3 nucleotides of the 5′ and/or 3′ termini. The sense strand need only be sufficiently complementary with the antisense strand to maintain the over all double stranded character of the molecule.

As discussed elsewhere herein, and in the material incorporated by reference in its entirety, an iRNA agent will often be modified or include nucleoside surrogates. Single stranded regions of an iRNA agent will often be modified or include nucleoside surrogates, e.g., the unpaired region or regions of a hairpin structure, e.g., a region which links two complementary regions, can have modifications or nucleoside surrogates. Modification to stabilize one or more 3′- or 5′-termini of an iRNA agent, e.g., against exonucleases, or to favor the antisense siRNA agent to enter into RISC are also envisioned. Modifications can include C3 (or C6, C7, C12) amino linkers, thiol linkers, carboxyl linkers, non-nucleotide spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

iRNA agents include: molecules that are long enough to trigger the interferon response (which can be cleaved by Dicer (Bernstein et al. 2001. Nature, 409:363-366) and enter a RISC(RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

Each strand of an siRNA agent can be equal to or less than 30, 25, 24, 23, 22, 21, or nucleotides in length. The strand may be at least 19 nucleotides in length. For example, each strand can be between 21 and 25 nucleotides in length. siRNA agents may have a duplex region of 17, 18, 19, 29, 21, 22, 23, 24, or 25 nucleotide pairs, and one or more overhangs, or one or two 3′ overhangs, of 2-3 nucleotides.

In addition to homology to target RNA and the ability to down regulate a target gene, an iRNA agent may have one or more of the following properties:
(1) it may be of the Formula VI set out in the RNA Agent section below;
(2) if single stranded it may have a 5′ modification which includes one or more phosphate groups or one or more analogs of a phosphate group;
(3) it may, despite modifications, even to a very large number, or all of the nucleosides, have an antisense strand that can present bases (or modified bases) in the proper three dimensional framework so as to be able to form correct base pairing and form a duplex structure with a homologous target RNA which is sufficient to allow down regulation of the target, e.g., by cleavage of the target RNA;
(4) it may, despite modifications, even to a very large number, or all of the nucleosides, still have “RNA-like” properties, i.e., it may possess the overall structural, chemical and physical properties of an RNA molecule, even though not exclusively, or even partly, of ribonucleotide-based content. For example, an iRNA agent can contain, e.g., a sense and/or an antisense strand in which all of the nucleotide sugars contain e.g., 2′ fluoro in place of 2′ hydroxyl. This deoxyribonucleotide-containing agent can still be expected to exhibit RNA-like properties. While not wishing to be bound by theory, the electronegative fluorine prefers an axial orientation when attached to the C2′ position of ribose. This spatial preference of fluorine can, in turn, force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. A modified moiety at the 2′ sugar position may be able to enter into H bonding which is more characteristic of the OH moiety of a ribonucleotide than the H moiety of a deoxyribonucleotide. Certain iRNA agents will: exhibit a C_{3′}-endo pucker in all, or at least 50, 75, 80, 85, 90, or 95% of its sugars; exhibit a C_{3′}-endo pucker in a sufficient amount of its sugars that it can give rise to a the RNA-characteristic A-family-type helix; will have no more than 20, 10, 5, 4, 3, 2, or 1 sugar which is not a C_{3′}-endo pucker structure. Regardless of the nature of the modification, and even though the RNA agent can contain deoxynucleotides or modified deoxynucleotides, particularly in overhang or other single strand regions, it is certain DNA molecules, or any molecule in which more than 50, 60, or 70% of the nucleotides in the molecule, or more than 50, 60, or 70% of the nucleotides in a duplexed region are deoxyribonucleotides, or modified deoxyribonucleotides which are deoxy at the 2′ position, are excluded from the definition of RNA agent.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. In certain embodiments single strand iRNA agents are 5′ phosphorylated or include a phosphoryl analog at the 5′ prime terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g., RP(OH)(O)—O-5′-, (OH)2(O)P-5′-CH2-), 5′-alkyletherphosphonates (R=alkylether=methoxymethyl (MeOCH2-), ethoxymethyl, etc., e.g., RP(OH)(O)—O-5′-). (These modifications can also be used with the antisense strand of a double stranded iRNA.)

A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

The antisense strand of a double stranded iRNA agent may be equal to or at least, 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The sense strand of a double stranded iRNA agent may be equal to or at least 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The double strand portion of a double stranded iRNA agent may be equal to or at least, 14, 15, 16 17, 18, 19, 20, 21, 22, 23, 24, 25, 29, 40, or 60 nucleotide pairs in length. It may be equal to or less than 200, 100, or 50, nucleotides pairs in length. Ranges may be 15-30, 17 to 23, 19 to 23, and 19 to 21 nucleotides pairs in length.

In many embodiments, the ds iRNA agent is sufficiently large that it can be cleaved by an endogenous molecule, e.g., by Dicer, to produce smaller ds iRNA agents, e.g., siRNAs agents.

The present invention further includes iRNA agents that target within the sequence targeted by one of the iRNA agents of the present invention. As used herein a second iRNA agent is said to target within the sequence of a first iRNA agent if the second iRNA agent cleaves the message anywhere within the mRNA that is complementary to the antisense strand of the first iRNA agent. Such a second agent will generally consist of at least 15 contiguous nucleotides coupled to additional nucleotide sequences taken from the region contiguous to the selected sequence in the target gene.

The dsiRNAs of the invention can contain one or more mismatches to the target sequence. In a preferred embodiment, the dsiRNA of the invention contains no more than 3 mismatches. If the antisense strand of the dsiRNA contains mismatches to a target sequence, it is preferable that the area of mismatch not be located in the center of the region of complementarity. If the antisense strand of the dsiRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide dsiRNA strand which is complementary to a region of the target gene, the dsRNA generally does not contain any mismatch within the central 13 nucleotides. The methods described within the invention can be used to determine whether a dsiRNA containing a mismatch to a target sequence is effective in inhibiting the expression of the target gene. Consideration of the efficacy of dsiRNAs with mismatches in inhibiting expression of the target gene may be important, especially if the particular region of complementarity in the target gene is known to have polymorphic sequence variation within the population.

In some embodiments, the sense-strand comprises a mismatch to the antisense strand. In some embodiments, the mismatch is at the 5 nucleotides from the 3′-end, for example 5, 4, 3, 2, or 1 nucleotide from the end of the region of complementarity. In some embodiments, the mismatch is located in the target cleavage site region. In one embodiment, the sense strand comprises no more than 1, 2, 3, 4 or 5 mismatches to the antisense strand. In preferred embodiments, the sense strand comprises no more than 3 mismatches to the antisense strand.

In certain embodiments, the sense strand comprises a nucleobase modification, e.g. an optionally substituted natural or non-natural nucleobase, a universal nucleobase, in the target cleavage site region.

The “target cleavage site” herein means the backbone linkage in the target gene, e.g. target mRNA, or the sense strand that is cleaved by the RISC mechanism by utilizing the iRNA agent. And the “target cleavage site region” comprises at least one or at least two nucleotides on both side of the cleavage site. For the sense strand, the target cleavage site is the backbone linkage in the sense strand that would get cleaved if the sense strand itself was the target to be cleaved by the RNAi mechanism. The target cleavage site can be determined using methods known in the art, for example the 5′-RACE assay as detailed in Soutschek et al., Nature (2004) 432, 173-178. As is well understood in the art, the cleavage site region for a conical double stranded RNAi agent comprising two 21-nucleotides long strands (wherein the strands form a double stranded region of 19 consective basepairs having 2-nucleotide single stranded overhangs at the 3′-ends), the cleavage site region corresponds to postions 9-12 from the 5′-end of the sense strand.

The present invention also includes nucleic acids which are chimeric compounds. “Chimeric” nucleic acid compounds or “chimeras,” in the context of this invention, are nucleic acid compounds, which contain two or more chemically distinct regions, each made up of at least one monomer unit, i.e., a nucleotide in the case of a nucleic acid compound. These nucleic acids typically contain at least one region wherein the nucleic acid is modified so as to confer upon the it increased resistance to nuclease degradation, increased cellular uptake, and/or increased binding affinity for the target nucleic acid. An additional region of the nucleic acid may serve as a substrate for enzymes capable of cleaving RNA:DNA or RNA:RNA hybrids. By way of example, RNase H is a cellular endonuclease which cleaves the RNA strand of an RNA:DNAduplex. Activation of RNase H, therefore, results in cleavage of the RNA target, thereby greatly enhancing the efficiency of dsRNA inhibition of gene expression.

The present invention also includes ds iRNAs wherein the two strands are linked together. The two strands can be linked together by a polynucleotide linker such as (dT)ₙ; wherein n is 4-10, and thus forming a hairpin. The two strands can also be linked together by a non-nucleosidic linker, e.g. a linker described herein. It will be appreciated by one of skill in the art that any oligonucleotide chemical modifications or variations describe herein can be used in the polynucleotide linker.

The double stranded oligonucleotides can be optimized for RNA interference by increasing the propensity of the duplex to disassociate or melt (decreasing the free energy of duplex association), in the region of the 5′ end of the antisense strand This can be accomplished, e.g., by the inclusion of modifications or modified nucleosides which increase the propensity of the duplex to disassociate or melt in the region of the 5′ end of the antisense strand. It can also be accomplished by inclusion of modifications or modified nucleosides or attachment of a ligand that increases the propensity of the duplex to disassociate of melt in the region of the 5′ end of the antisense strand. While not wishing to be bound by theory, the effect may be due to promoting the effect of an enzyme such as helicase, for example, promoting the effect of the enzyme in the proximity of the 5′ end of the antisense strand.

Modifications which increase the tendency of the 5′ end of the antisense strand in the duplex to dissociate can be used alone or in combination with other modifications described herein, e.g., with modifications which decrease the tendency of the 3′ end of the antisense in the duplex to dissociate. Likewise, modifications which decrease the tendency of the 3′ end of the antisense in the duplex to dissociate can be used alone or in combination with other modifications described herein, e.g., with modifications which increase the tendency of the 5′ end of the antisense in the duplex to dissociate.

Nucleic acid base pairs can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; I:C is preferred over G:C (I=inosine); mismatches, e.g., non-canonical or other than canonical pairings are preferred over canonical (A:T, A:U, G:C) pairings; pairings which include a universal base are preferred over canonical pairings.

It is preferred that pairings which decrease the propensity to form a duplex are used at 1 or more of the positions in the duplex at the 5′ end of the antisense strand. The terminal pair (the most 5′ pair in terms of the antisense strand), and the subsequent 4 base pairing positions (going in the 3′ direction in terms of the antisense strand) in the duplex are preferred for placement of modifications to decrease the propensity to form a duplex. More preferred are placements in the terminal most pair and the subsequent 3, 2, or 1 base pairings. It is preferred that at least 1, and more preferably 2, 3, 4, or 5 of the base pairs from the 5′-end of antisense strand in the duplex be chosen independently from the group of: A:U, G:U, I:C, mismatched pairs, e.g., non-canonical or other than canonical pairings or pairings which include a universal base. In a preferred embodiment at least one, at least 2, or at least 3 base-pairs include a universal base.

Modifications or changes which promote dissociation are preferably made in the sense strand, though in some embodiments, such modifications/changes will be made in the antisense strand.

Nucleic acid base pairs can also be ranked on the basis of their propensity to promote stability and inhibit dissociation or melting (e.g., on the free energy of association or dissociation of a particular pairing, the simplest approach is to examine the pairs on an individual pair basis, though next neighbor or similar analysis can also be used). In terms of promoting duplex stability: G:C is preferred over A:U, Watson-Crick matches (A:T, A:U, G:C) are preferred over non-canonical or other than canonical pairings, analogs that increase stability are preferred over Watson-Crick matches (A:T, A:U, G:C), e.g. 2-amino-A:U is preferred over A:U, 2-thio U or 5 Me-thio-U:A, are preferred over U:A, G-clamp (an analog of C having 4 hydrogen bonds):G is preferred over C:G, guanadinium-G-clamp:G is preferred over C:G, psuedo uridine:A, is preferred over U:A, sugar modifications, e.g., 2′ modifications, e.g., 2′F, ENA, or LNA, which enhance binding are preferred over non-modified moieties and can be present on one or both strands to enhance stability of the duplex.

It is preferred that pairings which increase the propensity to form a duplex are used at 1 or more of the positions in the duplex at the 3′ end of the antisense strand. The terminal pair (the most 3′ pair in terms of the antisense strand), and the subsequent 4 base pairing positions (going in the 5′ direction in terms of the antisense strand) in the duplex are preferred for placement of modifications to decrease the propensity to form a duplex. More preferred are placements in the terminal most pair and the subsequent 3, 2, or 1 base pairings. It is preferred that at least 1, and more preferably 2, 3, 4, or 5 of the pairs of the recited regions be chosen independently from the group of: G:C, a pair having an analog that increases stability over Watson-Crick matches (A:T, A:U, G:C), 2-amino-A:U, 2-thio U or 5 Me-thio-U:A, G-clamp (an analog of C having 4 hydrogen bonds):G, guanadinium-G-clamp:G, psuedo uridine:A, a pair in which one or both subunits has a sugar modification, e.g., a 2′ modification, e.g., 2′F, ENA, or LNA, which enhance binding. In some embodiments, at least one, at least, at least 2, or at least 3, of the base pairs promote duplex stability.

In a preferred embodiment at least one, at least 2, or at least 3, of the base pairs are a pair in which one or both subunits has a sugar modification, e.g., a 2′ modification, e.g., 2′-O-methyl, 2′-O-Me (2′-O-methyl), 2′-O-MOE (2′-O-methoxyethyl), 2′-F, 2′-O—CH₂-(4′-C) (LNA) and 2′-O—CH₂CH₂-(4′-C) (ENA), which enhances binding.

G-clamps and guanidinium G-clamps are discussed in the following references: Holmes and Gait, “The Synthesis of 2′-O-Methyl G-Clamp Containing Oligonucleotides and Their Inhibition of the HIV-1 Tat-TAR Interaction,” Nucleosides, Nucleotides & Nucleic Acids, 22:1259-1262, 2003; Holmes et al., “Steric inhibition of human immunodeficiency virus type-1 Tat-dependent trans-activation in vitro and in cells by oligonucleotides containing 2′-O-methyl G-clamp ribonucleoside analogues,” Nucleic Acids Research, 31:2759-2768, 2003; Wilds, et al., “Structural basis for recognition of guanosine by a synthetic tricyclic cytosine analogue: Guanidinium G-clamp,” Helvetica Chimica Acta, 86:966-978, 2003; Rajeev, et al., “High-Affinity Peptide Nucleic Acid Oligomers Containing Tricyclic Cytosine Analogues,” Organic Letters, 4:4395-4398, 2002; Ausin, et al., “Synthesis of Amino- and Guanidino-G-Clamp PNA Monomers,” Organic Letters, 4:4073-4075, 2002; Maier et al., “Nuclease resistance of oligonucleotides containing the tricyclic cytosine analogues phenoxazine and 9-(2-aminoethoxy)-phenoxazine (“G-clamp”) and origins of their nuclease resistance properties,” Biochemistry, 41:1323-7, 2002; Flanagan, et al., “A cytosine analog that confers enhanced potency to antisense oligonucleotides,” Proceedings Of The National Academy Of Sciences Of The United States Of America, 96:3513-8, 1999.

As is discussed above, ds iRNA can be modified to both decrease the stability of the antisense 5′ end of the duplex and increase the stability of the antisense 3′ end of the duplex. This can be effected by combining one or more of the stability decreasing modifications in the antisense 5′ end of the duplex with one or more of the stability increasing modifications in the antisense 3′ end of the duplex.

It may be desirable to modify one or both of the antisense and sense strands of a double strand iRNA agent. In some cases they will have the same modification or the same class of modification but in other cases the sense and antisense strand will have different modifications, e.g., in some cases it is desirable to modify only the sense strand. It may be desirable to modify only the sense strand, e.g., to inactivate it, e.g., the sense strand can be modified in order to inactivate the sense strand and prevent formation of an active siRNA/protein or RISC. This can be accomplished by a modification which prevents 5′-phosphorylation of the sense strand, e.g., by modification with a 5′-O-methyl ribonucleotide (see Nykänen et al., (2001) ATP requirements and small interfering RNA structure in the RNA interference pathway. Cell 107, 309-321.) Other modifications which prevent phosphorylation can also be used, e.g., simply substituting the 5′-OH by H rather than O-Me. Alternatively, a large bulky group may be added to the 5′-phosphate turning it into a phosphodiester linkage, though this may be less desirable as phosphodiesterases can cleave such a linkage and release a functional siRNA 5′-end. Antisense strand modifications include 5′ phosphorylation as well as any of the other 5′ modifications discussed herein, particularly the 5′ modifications discussed above in the section on single stranded iRNA molecules.

The sense and antisense strands may be chosen such that the ds iRNA agent includes a single strand or unpaired region at one or both ends of the molecule. Thus, a ds iRNA agent may contain sense and antisense strands, paired to contain an overhang, e.g., one or two 5′ or 3′ overhangs, or a 3′ overhang of 2-3 nucleotides. Many embodiments will have a 3′ overhang. Certain siRNA agents will have single-stranded overhangs, in some embodiments 3′ overhangs, of 1 or 2 or 3 nucleotides in length at each end. The overhangs can be the result of one strand being longer than the other, or the result of two strands of the same length being staggered. 5′ ends may be phosphorylated.

In one embodiment, the single-stranded overhang has the sequence 5′-GCNN-3′, wherein N is independently for each occuurence, A, G, C, U, dT, dU or absent. Double-stranded iRNA having only one overhang has proven particularly stable and effective in vivo, as well as in a variety of cells, cell culture mediums, blood, and serum. The dsRNA may also have a blunt end, generally located at the 5′-end of the antisense strand.

In one embodiment, the antisense strand of the ds iRNA has 1-10 nucleotides overhangs each at the 3′ end and the 5′ end over the sense strand. In one embodiment, the sense strand of the ds iRNA has 1-10 nucleotides overhangs each at the 3′ end and the 5′ end over the antisense strand.

In some embodiments, the length for the duplexed region is between 15 and 30, or 18, 19, 20, 21, 22, and 23 nucleotides in length, e.g., in the siRNA agent range discussed above. siRNA agents can resemble in length and structure the natural Dicer processed products from long dsiRNAs. Embodiments in which the two strands of the siRNA agent are linked, e.g., covalently linked are also included. Hairpin, or other single strand structures which provide the required double stranded region, and a 3′ overhang are also within the invention.

In some embodiments, the length for the duplexed region is between 10-15, e.g. 10, 11, 12, 13, 14 and 15 nucletoides in length and the antisense strand has 1-10 nucleotides single-strand overhangs each at the 3′ end and the 5′ end over the sense strand.

The isolated iRNA agents described herein, including ds iRNA agents and siRNA agents can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an siRNA agent of 21 to 23 nucleotides.

As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In one embodiment, an iRNA agent is “sufficiently complementary” to a target RNA, e.g., a target mRNA, such that the iRNA agent silences production of protein encoded by the target mRNA. In another embodiment, the iRNA agent is “exactly complementary” to a target RNA, e.g., the target RNA and the iRNA agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the iRNA agent specifically discriminates a single-nucleotide difference. In this case, the iRNA agent only mediates RNAi if exact complementary is found in the region (e.g., within 7 nucleotides of) the single-nucleotide difference.

As used herein, the term “oligonucleotide” refers to a nucleic acid molecule (RNA or DNA) for example of length less than 100, 200, 300, or 400 nucleotides.

RNA agents discussed herein include unmodified RNA as well as RNA which have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. The art has often referred to rare or unusual, but naturally occurring, RNAs as modified RNAs, see, e.g., Limbach et al., (1994) Summary: the modified nucleosides of RNA, Nucleic Acids Res. 22: 2183-2196. Such rare or unusual RNAs, often termed modified RNAs (apparently because the are typically the result of a post transcriptionally modification) are within the term unmodified RNA, as used herein. Modified RNA refers to a molecule in which one or more of the components of the nucleic acid, namely sugars, bases, and phosphate moieties, are different from that which occur in nature, for example, different from that which occurs in the human body. While they are referred to as modified “RNAs,” they will of course, because of the modification, include molecules which are not RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. Examples of all of the above are discussed herein.

Much of the discussion below refers to single strand molecules. In many embodiments of the invention a double stranded iRNA agent, e.g., a partially double stranded iRNA agent, is envisioned. Thus, it is understood that that double stranded structures (e.g., where two separate molecules are contacted to form the double stranded region or where the double stranded region is formed by intramolecular pairing (e.g., a hairpin structure)) made of the single stranded structures described below are within the invention. Lengths are described elsewhere herein.

As nucleic acids are polymers of subunits, many of the modifications described below occur at a position which is repeated within a nucleic acid, e.g., a modification of a base, or a phosphate moiety, or the a non-linking O of a phosphate moiety. In some cases the modification will occur at all of the subject positions in the nucleic acid but in many cases it will not. By way of example, a modification may only occur at a 3′ or 5′ terminal position, may only occur in a terminal regions, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. The 5′ end or ends can be phosphorylated.

A modification described herein may be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

In some embodiments it is possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

### The Phosphate Group

The phosphate group is a negatively charged species. The charge is distributed equally over the two non-linking oxygen atoms (i.e., X and Y in Formula VI above). However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. Unlike the situation where only one of X or Y is altered, the phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species. Thus, while not wishing to be bound by theory, modifications to both X and Y which eliminate the chiral center, e.g., phosphorodithioate formation, may be desirable in that they cannot produce diastereomer mixtures. Thus, X can be any one of S, Se, B, BR₃ (R is hydrogen, alkyl, aryl), C (i.e. an alkyl group, an aryl group, etc. . . . ), H, NR₂ (R is hydrogen, alkyl, aryl, etc.), or OR (R is alkyl or aryl). Thus Y can be any one of S, Se, B, BR₃ (R is hydrogen, alkyl, aryl), C (i.e. an alkyl group, an aryl group, etc. . . . ), H, NR₂ (R is hydrogen, alkyl, aryl, etc. . . . ), or OR (R is alkyl or aryl). Replacement of X and/or Y with sulfur is possible.

When the modification of the phosphate leads to phosphorous atom becoming stereogenic, such chiral phosphate can posses either the “R” configuration (herein Rp) or the “S” configuration (herein Sp).

The phosphate linker can also be modified by replacement of a linking oxygen (i.e., W or Z in Formula VI) with nitrogen (bridged phosphoroamidates), sulfur (bridged phosphorothioates) and carbon (bridged methylenephosphonates). The replacement can occur at a terminal oxygen (position W (3′) or position Z (5′). Replacement of W with carbon or Z with nitrogen is possible. When the bridging oxygen is 3′-oxygen of a nucleoside, replacement with carbon is preferred. When the bridging oxygen is the 5′-oxygen of a nucleoside, replacement with nitrogen is preferred.

Candidate agents can be evaluated for suitability as described below.

### The Sugar Group

A modified RNA can include modification of all or some of the sugar groups of the ribonucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′ alkoxide ion. The 2′ alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; ENA in which the 2′ hydroxyl is connected by a ethylene bridge, to the 4′ carbon of the same ribose sugar; O-AMINE (AMINE=NH₂, alkylamino, dialkylamino, heterocyclyl, arylamino, diaryl amino, heteroaryl amino, diheteroaryl amino, ethylene diamine, polyamino and aminoalkoxy), O(CH₂)ₙAMINE, (e.g., AMINE=NH₂, alkylamino, dialkylamino, heterocyclyl, arylamino, diaryl amino, heteroaryl amino, diheteroaryl amino, ethylene diamine, polyamino and aminoalkoxy). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e., deoxyribose sugars, which are of particular relevance to the overhang portions of partially ds RNA); halo (e.g., fluoro); amino (e.g., NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, or ureido. Other substitutents of certain embodiments include 2′-methoxyethyl, 2′-OCH3,2′-O-allyl, 2′-C— allyl, and 2′-fluoro.

Other preferred substitutents are 2′-O-[2-(methylamino)-2-oxoethyl] (2′-O-NMA), 2′—NH₂, 2′-O-amine, 2′-SH, 2′-S-alkyl, 2′-S-allyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-β-dimethylaminopropyl (2′-O-DMAP) and 2′-O-dimethylaminoethyloxyethyl (2′-O-DMAEOE).

In some embodiments, the 2′- and the 4′-carbons of the same ribose sugar may be linked together by a linker described herein.

The sugar group can also contain one or more carbons that possess the opposite stereochemical configuration than that of the corresponding carbon in ribose. Thus, a modified RNA can include nucleotides containing e.g., arabinose, as the sugar.

The sugar group can also have an alpha linkage at the 1′ position on the sugar, e.g., alpha-nucleosides.

The sugar group can also be a L-sugar, e.g. L-nucleosides.

Modified RNA's can also include “abasic” sugars, which lack a nucleobase at C-1′. These abasic sugars can also be further contain modifications at one or more of the constituent sugar atoms.

To maximize nuclease resistance, the 2′ modifications can be used in combination with one or more phosphate linker modifications (e.g., phosphorothioate). The so-called “chimeric” oligonucleotides are those that contain two or more different modifications.

Candidate modifications can be evaluated as described below.

### Replacement of the Phosphate Group

The phosphate group can be replaced by non-phosphorus containing connectors (cf. Bracket I in Formula VI above). While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In certain embodiments, replacements may include the methylenecarbonylamino and methylenemethylimino groups.

Candidate modifications can be evaluated as described below.

### Replacement of Ribophosphate Backbone

Oligonucleotide-mimicking scaffolds can also be constructed wherein the phosphate linker and ribose sugar are replaced by nuclease resistant nucleoside or nucleotide surrogates (see Bracket II of Formula I above). While not wishing to be bound by theory, it is believed that the absence of a repetitively charged backbone diminishes binding to proteins that recognize polyanions (e.g., nucleases). Again, while not wishing to be bound by theory, it can be desirable in some embodiment, to introduce alterations in which the bases are tethered by a neutral surrogate backbone.

Examples include the mophilino, cyclobutyl, pyrrolidine and peptide nucleic acid (PNA) nucleoside surrogates. In certain embodiments, PNA surrogates may be used.

Modified phosphate linkages where at least one of the oxygens linked to the phosphate has been replaced or the phosphate group has been replaced by a non-phosphorous group, are also referred to as “non-phosphodiester backbone linkage.”

Preferred backbone modifications are phsophorothioate, phosphorodithioate, phosphoramidate, phosphonate, alkylphosphonate, siloxane, carbonate, carboxymethyl, carbamate, amide, thioether, ethylene oxide linker, sulfonate, sulfonamide, thioformacetal, formacetal, oxime, methyleneimino, methyleneaminocarbonyl, methylenemethylimino (MMI), methylenehydrazo, methylenedimethylhydrazo (MDH) and methyleneoxymethylimino.

Candidate modifications can be evaluated as described below.

### Types of Backbone Linkages

The canonical 3′-5′ backbone linkage can also be replaced with linkage between other positions on the nucleosides. In some embodiments, the oligonucleotide comprises at least one of 5′-5′,3′-3′,3′-2′,2′-3′,2′-3′ or 2′-5′ backbone linkage.

In some embodiments, the last nucleotide on the end of the oligonucleotide is linked via a 5′-5′,3′-3′,3′-2′,2′-3′ or 2′-3′ backbone linkage to the rest of the oligonucleotide.

### Terminal Modifications

The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The terminal atom of the spacer can connect to or replace the linking atom of the phosphate group or the C-3′ or C-5′ O, N, S or C group of the sugar. Alternatively, the spacer can connect to or replace the terminal atom of a nucleotide surrogate (e.g., PNAs). These spacers or linkers can include e.g., —(CH₂)ₙ—, —(CH₂)ₙN—, —(CH₂)ₙ—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents. When a spacer/phosphate-functional molecular entity-spacer/phosphate array is interposed between two strands of iRNA agents, this array can substitute for a hairpin RNA loop in a hairpin-type RNA agent. The 3′ end can be an —OH group. While not wishing to be bound by theory, it is believed that conjugation of certain moieties can improve transport, hybridization, and specificity properties. Again, while not wishing to be bound by theory, it may be desirable to introduce terminal alterations that improve nuclease resistance. Other examples of terminal modifications include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can be added for a number of reasons, including as discussed elsewhere herein to modulate activity or to modulate resistance to degradation. Terminal modifications useful for modulating activity include modification of the 5′ end with phosphate or phosphate analogs. E.g., in certain embodiments iRNA agents, especially antisense strands, are 5′ phosphorylated or include a phosphoryl analog at the 5′ prime terminus. 5′-phosphate modifications include those which are compatible with RISC mediated gene silencing. Suitable modifications include: 5′-monophosphate ((HO)2(O)P—O-5); 5′-diphosphate ((HO)2(O)P—O—P(HO)(O)—O-5′); 5′-triphosphate ((HO)2(O)P—)-(HO)(O)P—O—P(HO)(O)—O-5′); 5′-guanosine cap (7-methylated or non-methylated) (7m-G-O-5′-(H0)(O)P—O—(HO)(O)P—O—P(HO)(O)—O-5′); 5′-adenosine cap (Appp), and any modified or unmodified nucleotide cap structure (N—O-5′-(H0)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g., RP(OH)(O)—O-5′-, (OH)2(O)P-5′-CH2-), 5′-alkyletherphosphonates (R=alkylether=methoxymethyl (MeOCH2-), ethoxymethyl, etc., e.g., RP(OH)(O)—O-5′-).

Terminal modifications can also be useful for monitoring distribution, and in such cases the groups to be added may include fluorophores, e.g., fluorscein or an Alexa dye, e.g., Alexa 488. Terminal modifications can also be useful for enhancing uptake, useful modifications for this include cholesterol. Terminal modifications can also be useful for cross-linking an RNA agent to another moiety; modifications useful for this include mitomycin C.

Candidate modifications can be evaluated as described below.

### The Bases

Adenine, guanine, cytosine and uracil are the most common bases found in RNA. These bases can be modified or replaced to provide RNA's having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed. Examples include 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N-6-isopentenyladenine, N-methylguanines, or O-alkylated bases. Further purines and pyrimidines include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

Generally, base changes are not used for promoting stability, but they can be useful for other reasons, e.g., some, e.g., 2,6-diaminopurine and 2 amino purine, are fluorescent. Modified bases can reduce target specificity. This may be taken into consideration in the design of iRNA agents.

In some embodiments, nucleobase is chosen from a group consisting of inosine, thymine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

Candidate modifications can be evaluated as described below.

### Cationic Groups

Modifications to oligonucleotides can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

Placement of Modifications within an Oligonucleotide

Some modifications may preferably be included on an oligonucleotide at a particular location, e.g., at an internal position of a strand, or on the 5′ or 3′ end of an oligonucleotide. A preferred location of a modification on an oligonucleotide, may confer preferred properties on the agent. For example, preferred locations of particular modifications may confer optimum gene silencing properties, or increased resistance to endonuclease or exonuclease activity.

One or more nucleotides of an oligonucleotide may have a 2′-5′ linkage. One or more nucleotides of an oligonucleotide may have inverted linkages, e.g. 3′-3′,3′-2′,5′-5′, 2′-2′ or 2′-3′ linkages.

An oligonucleotide may comprise at least one 5′-pyrimidine-purine-3′ (5′-PyPu-3′) dinucleotide wherein the pyrimidine is modified with a modification chosen independently from a group consisting of 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA) and 2′-O—CH₂CH₂-(4′-C) (ENA).

In one embodiment, the 5′-most pyrimidines in all occurrences of sequence motif 5′-pyrimidine-purine-3′ (5′-PyPu-3′) dinucleotide in the oligonucleotide are modified with a modification chosen from a group consisting of 2″-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA) and 2′-O—CH₂CH₂-(4′-C) (ENA).

A double-stranded oligonucleotide may include at least one 5′-uridine-adenine-3′ (5′-UA-3′) dinucleotide wherein the uridine is a 2′-modified nucleotide, or a 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide, wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. Double-stranded oligonucleotides including these modifications are particularly stabilized against endonuclease activity.

### Evaluation of Candidate RNAs

One can evaluate a candidate RNA agent, e.g., a modified RNA, for a selected property by exposing the agent or modified molecule and a control molecule to the appropriate conditions and evaluating for the presence of the selected property. For example, resistance to a degradent can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dsiRNA agents.

In an alternative functional assay, a candidate dsiRNA agent homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dsiRNA agent would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### GENERAL REFERENCES

The oligoribonucleotides and oligoribonucleosides used in accordance with this invention may be with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotide- s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein.

### DEFINITIONS

The term “copolymer” means a polymer derived from more than one species of monomer.

The term “random copolymer” means a copolymer consisting of macromolecules in which the sequential distribution of the monomeric units obeys known statistical laws, e.g. the sequential distribution of monomer units follows Markovian statistics.

The term “block copolymer” means a polymer composed of macromolecules consisting of a linear sequence of blocks, wherein the term “block” means a portion of macromolecule comprising many constitutional units that has at least one feature that is not present in the adjacent portions.

The term “polymer matrix” refers to all of the polymer layers or sublayers on the metal surface. This can include activating, first, additional, and/or barrier layers.

The term “amphiphilic copolymer” means a polymer containing both hydrophilic (water-soluble) and hydrophobic (water-insoluble) segments.

The terms “silence” and “inhibit the expression of” and related terms and phrases, refer to the at least partial suppression of the expression of a gene targeted by an siRNA or siNA, as manifested by a reduction of the amount of mRNA transcribed from the target gene which may be isolated from a first cell or group of cells in which the target gene is transcribed and which has or have been treated such that the expression of the target gene is inhibited, as compared to a second cell or group of cells substantially identical to the first cell or group of cells but which has or have not been so treated (i.e., control cells).

The term “phosphorous containing linkage” include any linkage with a phosphorus atom included, such as natural phosphate, phosphorothioate, phosphorodithioate, borano phosphate, borano thiophospahte, phosphonate, halogen substituted phosphoantes, phosphoramidates, phosphodiester, phosphotriester, thiophosphodiester, thiophosphotriester, diphosphates and triphosphates.

The phosphours containing linkage can be optionally protected. Representative protecting groups for phosphorus containing linkages such as phosphodiester and phosphorothioate linkages include β-cyanoethyl, diphenylsilylethyl, δ-cyanobutenyl, cyano p-xylyl (CPX), N-methyl-N-trifluoroacetyl ethyl (META), acetoxy phenoxy ethyl (APE) and butene-4-yl groups. See for example U.S. Pat. Nos. 4,725,677 and Re. 34,069 (β-cyanoethyl); Beaucage, S. L. and Iyer, R. P., Tetrahedron, 49 No. 10, pp. 1925-1963 (1993); Beaucage, S. L. and Iyer, R. P., Tetrahedron, 49 No. 46, pp. 10441-10488 (1993); Beaucage, S. L. and Iyer, R. P., Tetrahedron, 48 No. 12, pp. 2223-2311 (1992).

The term “halo” or “halogen” refers to any radical of fluorine, chlorine, bromine or iodine.

The term “aliphatic,” as used herein, refers to a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines. Aliphatic groups as used herein may optionally include further substitutent groups.

The term “acyl” refers to hydrogen, alkyl, partially saturated or fully saturated cycloalkyl, partially saturated or fully saturated heterocycle, aryl, and heteroaryl substituted carbonyl groups. For example, acyl includes groups such as (Ci-C6)alkanoyl (e.g., formyl, acetyl, propionyl, butyryl, valeryl, caproyl, t-butylacetyl, etc.), (C3-Ce)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, C₁-C₁₀ indicates that the group may have from 1 to 10 (inclusive) carbon atoms in it. The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene. The term “aminoalkyl” refers to an alkyl substituted with an amino. The term “mercapto” refers to an —SH radical. The term “thioalkoxy” refers to an —S-alkyl radical.

The term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” or “heterocyclic” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, aryl, heterocyclyl, or heteroaryl group at any atom of that group. Suitable substituents include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, ureido or conjugate groups.

In many cases, protecting groups are used during preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, for example, are disclosed by Beaucage et al. (Tetrahedron 1992, 48, 2223-2311). Further hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y., 1991.

Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p′-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

### MicroRNAs

MicroRNAs (miRNAs or mirs) are a highly conserved class of small RNA molecules that are transcribed from DNA in the genomes of plants and animals, but are not translated into protein. Pre-microRNAs are processed into miRNAs. Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S, NAR, 2004, 32, Database Issue, D109-D111.

Single-stranded oligonucleotides, including those described and/or identified as microRNAs or mirs which may be used as targets or may serve as a template for the design of oligonucleotides of the invention are taught in, for example, Esau, et al. US Publication No. 20050261218 (U.S. Ser. No. 10/909,125) entitled “Oligomeric compounds and compositions for use in modulation small non-coding RNAs” the entire contents of which is incorporated herein by reference. It will be appreciated by one of skill in the art that any oligonucleotide chemical modifications or variations describe herein also apply to single stranded oligonucleotides.

miRNA Mimics

miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs). miRNA mimics can be comprised of nucleic acid (modified or modified nucleic acids) including oligonucleotides comprising, without limitation, RNA, modified RNA, DNA, modified DNA, locked nucleic acids, or 2′-O,4′-C-ethylene-bridged nucleic acids (ENA), or any combination of the above (including DNA-RNA hybrids). In addition, miRNA mimics can comprise conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can comprise 2′ modifications (including 2′-O methyl modifications and 2′ F modifications) on one or both strands of the molecule and internucleotide modifications (e.g. phorphorthioate modifications) that enhance nucleic acid stability and/or specificity. In addition, miRNA mimics can include overhangs. The overhangs can consist of 1-6 nucleotides on either the 3′ or 5′ end of either strand and can be modified to enhance stability or functionality. In one embodiment, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

### Supermirs

A supermir refers to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target. This term includes oligonucleotides composed of naturally-occurring nucleobases, sugars and covalent internucleoside (backbone) linkages and which contain at least one non-naturally-occurring portion which functions similarly. Such modified or substituted oligonucleotides are preferred over native forms because of desirable properties such as, for example, enhanced cellular uptake, enhanced affinity for nucleic acid target and increased stability in the presence of nucleases. In a preferred embodiment, the supermir does not include a sense strand, and in another preferred embodiment, the supermir does not self-hybridize to a significant extent. An supermir featured in the invention can have secondary structure, but it is substantially single-stranded under physiological conditions. An supermir that is substantially single-stranded is single-stranded to the extent that less than about 50% (e.g., less than about 40%, 30%, 20%, 10%, or 5%) of the supermir is duplexed with itself. The supermir can include a hairpin segment, e.g., sequence, preferably at the 3′ end can self hybridize and form a duplex region, e.g., a duplex region of at least 1, 2, 3, or 4 and preferably less than 8, 7, 6, or n nucleotides, e.g., 5 nucleotides. The duplexed region can be connected by a linker, e.g., a nucleotide linker, e.g., 3, 4, 5, or 6 dTs, e.g., modified dTs. In another embodiment the supermir is duplexed with a shorter oligo, e.g., of 5, 6, 7, 8, 9, or 10 nucleotides in length, e.g., at one or both of the 3′ and 5′ end or at one end and in the non-terminal or middle of the supermir.

Antimir or miRNA Inhibitor

The terms “antimir” “microRNA inhibitor”, “miR inhibitor”, or “inhibitor” are synonymous and refer to oligonucleotides or modified oligonucleotides that interfere with the ability of specific miRNAs. In general, the inhibitors are nucleic acid or modified nucleic acids in nature including oligonucleotides comprising RNA, modified RNA, DNA, modified DNA, locked nucleic acids (LNAs), or any combination of the above. Modifications include 2′ modifications (including 2′-0 alkyl modifications and 2′ F modifications) and internucleotide modifications (e.g. phosphorothioate modifications) that can affect delivery, stability, specificity, intracellular compartmentalization, or potency. In addition, miRNA inhibitors can comprise conjugates that can affect delivery, intracellular compartmentalization, stability, and/or potency Inhibitors can adopt a variety of configurations including single stranded, double stranded (RNA/RNA or RNA/DNA duplexes), and hairpin designs, in general, microRNA inhibitors comprise contain one or more sequences or portions of sequences that are complementary or partially complementary with the mature strand (or strands) of the miRNA to be targeted, in addition, the miRNA inhibitor may also comprise additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

U1 adaptors

U1 adaptors inhibit polyA sites and are bifunctional oligonucleotides with a target domain complementary to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant Mol Biol 49:77-95). Nucleotides 2-11 of the 5′ end of U1 snRNA base pair bind with the 5′ ss of the pre mRNA. In one embodiment, oligonucleotides of the invention are U1 adaptors. In one embodiment, the U1 adaptor can be administered in combination with at least one other iRNA agent.

### Antagomirs

Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein, in its entirety. Antagomir RNAs may be synthesized using standard solid phase oligonucleotide synthesis protocols. See U.S. patent application Ser. Nos. 11/502,158 and 11/657,341 (the disclosure of each of which are incorporated herein by reference). An antagomir can include ligand-conjugated monomer subunits and monomers for oligonucleotide synthesis. Exemplary monomers are described in U.S. application Ser. No. 10/916,185, filed on Aug. 10, 2004. An antagomir can have a ZXY structure, such as is described in PCT Application No. PCT/US2004/07070 filed on Mar. 8, 2004. An antagomir can be complexed with an amphipathic moiety. Exemplary amphipathic moieties for use with oligonucleotide agents are described in PCT Application No. PCT/US2004/07070, filed on Mar. 8, 2004.

Antagomirs may be single stranded, double stranded, partially double stranded or hairpin-structured, chemically modified oligonucleotides that target a microRNA. An antagomir may consist essentially of or comprise about 12 or more contiguous nucleotides substantially complementary to an endogenous miRNA, and more particularly, agents that include about 12 or more contiguous nucleotides substantially complementary to a target sequence of an miRNA or pre-miRNA nucleotide sequence. In certain embodiments, an antagomir featured in the invention includes a nucleotide sequence sufficiently complementary to hybridize to a miRNA target sequence of about 12 to 25 nucleotides, in some instances about 15 to 23 nucleotides.

### Decoy Oligonucleotides

Because transcription factors can recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor. Examples of the utilization of decoy oligonucleotides may be found in Mann et al., J. Clin. Invest., 2000, 106: 1071-1075, which is expressly incorporated by reference herein, in its entirety.

An oligonucleotide agent featured in the invention can also be a decoy nucleic acid, e.g., a decoy RNA. A decoy nucleic acid resembles a natural nucleic acid, but may be modified in such a way as to inhibit or interrupt the activity of the natural nucleic acid. For example, a decoy RNA can mimic the natural binding domain for a ligand. The decoy RNA, therefore, competes with natural binding domain for the binding of a specific ligand. The natural binding target can be an endogenous nucleic acid, e.g., a pre-miRNA, miRNA, pre-mRNA, mRNA or DNA. For example, it has been shown that over-expression of HIV trans-activation response (TAR) RNA can act as a “decoy” and efficiently bind HIV tat protein, thereby preventing it from binding to TAR sequences encoded in the HIV RNA. In certain embodiments, a decoy RNA may include a modification that improves targeting, e.g., a targeting modification described herein.

### Antisense Oligonucleotides

Antisense oligonucleotides are single strands of DNA or RNA that are at least partially complementary to a chosen sequence. In the case of antisense RNA, they prevent translation of complementary RNA strands by binding to it. Antisense DNA can also be used to target a specific, complementary (coding or non-coding) RNA. If binding takes place, the DNA/RNA hybrid can be degraded by the enzyme RNase H. Examples of the utilization of antisense oligonucleotides may be found in Dias et al., Mol. Cancer. Ther., 2002, 1: 347-355, which is expressly incorporated by reference herein, in its entirety.

The single-stranded oligonucleotide agents featured in the invention include antisense nucleic acids. An “antisense” nucleic acid includes a nucleotide sequence that is complementary to a “sense” nucleic acid encoding a gene expression product, e.g., complementary to the coding strand of a double-stranded cDNA molecule or complementary to an RNA sequence, e.g., a pre-mRNA, mRNA, miRNA, or pre-miRNA. Accordingly, an antisense nucleic acid may form hydrogen bonds with a sense nucleic acid target.

Given a coding strand sequence (e.g., the sequence of a sense strand of a cDNA molecule), antisense nucleic acids can be designed according to the rules of Watson and Crick base pairing. The antisense nucleic acid molecule can be complementary to a portion of the coding or noncoding region of an RNA, e.g., a pre-mRNA or mRNA. For example, the antisense oligonucleotide can be complementary to the region surrounding the translation start site of a pre-mRNA or mRNA, e.g., the 5′ UTR. An antisense oligonucleotide can be, for example, about 10 to 25 nucleotides in length (e.g., about 11, 12, 13, 14, 15, 16, 18, 19, 20, 21, 22, 23, or 24 nucleotides in length). An antisense oligonucleotide can also be complementary to a miRNA or pre-miRNA.

In certain embodiments, an antisense nucleic acid can be constructed using chemical synthesis and/or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and target nucleic acids, e.g., phosphorothioate derivatives and acridine substituted nucleotides can be used. Other appropriate nucleic acid modifications are described herein. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

An antisense agent can include ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA, and prevent access of the translation machinery to the target RNA transcript, thereby preventing protein synthesis. An antisense molecule including only deoxyribonucleotides, or deoxyribonucleotides and ribonucleotides, e.g., DNA sequence flanked by RNA sequence at the 5′ and 3′ ends of the antisense agent, can hybridize to a complementary RNA, and the RNA target can be subsequently cleaved by an enzyme, e.g., RNAse H. Degradation of the target RNA prevents translation. The flanking RNA sequences can include 2′-O-methylated nucleotides, and phosphorothioate linkages, and the internal DNA sequence can include phosphorothioate internucleotide linkages. In some embodiments, the internal DNA sequence may be at least five nucleotides in length when targeting by RNAseH activity is desired.

For increased nuclease resistance, an antisense agent can be further modified by inverting the nucleoside at the 3′-terminus with a 3′-3′ linkage. In another alternative, the 3′-terminus can be blocked with an aminoalkyl group.

In other embodiments, an antisense oligonucleotide agent may include a modification that improves targeting, e.g., a targeting modification described herein.

### Aptamers

Aptamers are nucleic acid molecules that bind a specific target molecule or molecules. Aptamers may be RNA or DNA based, and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule.

An oligonucleotide agent featured in the invention can be an aptamer. An aptamer binds to a non-nucleic acid ligand, such as a small organic molecule or protein, e.g., a transcription or translation factor, and subsequently modifies (e.g., inhibits) activity. An aptamer can fold into a specific structure that directs the recognition of the targeted binding site on the non-nucleic acid ligand. An aptamer can contain any of the modifications described herein.

Ribozymes are oligonucleotides having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Methods of producing a ribozyme targeted to any target sequence are known in the art. Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

### Physiological Effects

The iRNA agents described herein can be designed such that determining therapeutic toxicity is made easier by the complementarity of the iRNA agent with both a human and a non-human animal sequence. By these methods, an iRNA agent can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the iRNA agent could be complementary to sequences within homologous genes, e.g., oncogenes or tumor suppressor genes, of the non-human mammal and the human. By determining the toxicity of the iRNA agent in the non-human mammal, one can extrapolate the toxicity of the iRNA agent in a human. For a more strenuous toxicity test, the iRNA agent can be complementary to a human and more than one, e.g., two or three or more, non-human animals.

The methods described herein can be used to correlate any physiological effect of an iRNA agent on a human, e.g., any unwanted effect, such as a toxic effect, or any positive, or desired effect.

Increasing Cellular Uptake of dsiRNAs

A method of the invention that includes administering an iRNA agent and a drug that affects the uptake of the iRNA agent into the cell. The drug can be administered before, after, or at the same time that the iRNA agent is administered. The drug can be covalently linked to the iRNA agent. The drug can be, for example, a lipopolysaccharid, an activator of p38 MAP kinase, or an activator of NF-κB. The drug can have a transient effect on the cell.

The drug can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The drug can also increase the uptake of the iRNA agent into the cell by activating an inflammatory response, for example. Exemplary drug's that would have such an effect include tumor necrosis factor alpha (TNFalpha), interleukin-1 beta, or gamma interferon.

### Organic Synthesis

An iRNA can be made by separately synthesizing each respective strand of a double-stranded RNA molecule. The component strands can then be annealed.

A large bioreactor, e.g., the OligoPilot II from Pharmacia Biotec AB (Uppsala Sweden), can be used to produce a large amount of a particular RNA strand for a given iRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the iRNA. Typically, the two complementary strands are produced separately and then annealed, e.g., after release from the solid support and deprotection.

Organic synthesis can be used to produce a discrete iRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage

iRNAs can also be made by cleaving a larger ds iRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be used. In one embodiment, RNA generated by this method is carefully purified to remove endotoxins that may contaminate preparations of the recombinant enzymes.

In vitro cleavage. dsiRNA is cleaved in vitro into iRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9. and Hammond Science 2001 Aug. 10; 293(5532):1146-50.

dsiRNA cleavage generally produces a plurality of iRNA species, each being a particular 21 to 23 nt fragment of a source dsiRNA molecule. For example, iRNAs that include sequences complementary to overlapping regions and adjacent regions of a source dsiRNA molecule may be present.

Regardless of the method of synthesis, the iRNA preparation can be prepared in a solution (e.g., an aqueous and/or organic solution) that is appropriate for formulation. For example, the iRNA preparation can be precipitated and redissolved in pure double-distilled water, and lyophilized. The dried iRNA can then be resuspended in a solution appropriate for the intended formulation process.

### Formulation

The iRNA agents described herein can be formulated for administration to a subject

For ease of exposition the formulations, compositions and methods in this section are discussed largely with regard to unmodified iRNA agents. It may be understood, however, that these formulations, compositions and methods can be practiced with other iRNA agents, e.g., modified iRNA agents, and such practice is within the invention.

A formulated iRNA composition can assume a variety of states. In some examples, the composition is at least partially crystalline, uniformly crystalline, and/or anhydrous (e.g., less than 80, 50, 30, 20, or 10% water). In another example, the iRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the iRNA composition is formulated in a manner that is compatible with the intended method of administration (see, below).

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

A iRNA preparation can be formulated in combination with another agent, e.g., another therapeutic agent or an agent that stabilizes a iRNA, e.g., a protein that complexes with iRNA to form an iRNP. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the iRNA preparation includes another iRNA agent, e.g., a second iRNA that can mediated RNAi with respect to a second gene, or with respect to the same gene. Still other preparation can include at least 3, 5, ten, twenty, fifty, or a hundred or more different iRNA species. Such iRNAs can mediated RNAi with respect to a similar number of different genes.

In one embodiment, the iRNA preparation includes at least a second therapeutic agent (e.g., an agent other than an RNA or a DNA). For example, a iRNA composition for the treatment of a viral disease, e.g., HIV, might include a known antiviral agent (e.g., a protease inhibitor or reverse transcriptase inhibitor). In another example, a iRNA composition for the treatment of a cancer might further comprise a chemotherapeutic agent.

Exemplary formulations are discussed below:

Micelles and other Membranous Formulations

For ease of exposition the micelles and other formulations, compositions and methods in this section are discussed largely with regard to unmodified iRNA agents. It may be understood, however, that these micelles and other formulations, compositions and methods can be practiced with other iRNA agents, e.g., modified iRNA agents, and such practice is within the invention. The iRNA agent, e.g., a double-stranded iRNA agent, or siRNA agent, (e.g., a precursor, e.g., a larger iRNA agent which can be processed into a siRNA agent, or a DNA which encodes an iRNA agent, e.g., a double-stranded iRNA agent, or siRNA agent, or precursor thereof)) composition can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the iRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linoleic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the iRNA composition and at least the alkali metal alkyl sulphate. The first micellar composition is then mixed with at least three micelle forming compounds to form a mixed micellar composition. In another method, the micellar composition is prepared by mixing the iRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### Particles

For ease of exposition the particles, formulations, compositions and methods in this section are discussed largely with regard to unmodified iRNA agents. It may be understood, however, that these particles, formulations, compositions and methods can be practiced with other iRNA agents, e.g., modified iRNA agents, and such practice is within the invention. In another embodiment, an iRNA agent, e.g., a double-stranded iRNA agent, or siRNA agent, (e.g., a precursor, e.g., a larger iRNA agent which can be processed into a siRNA agent, or a DNA which encodes an iRNA agent, e.g., a double-stranded iRNA agent, or siRNA agent, or precursor thereof) preparations may be incorporated into a particle, e.g., a microparticle. Microparticles can be produced by spray-drying, but may also be produced by other methods including lyophilization, evaporation, fluid bed drying, vacuum drying, or a combination of these techniques. See below for further description.

Sustained-Release Formulations. An iRNA agent, e.g., a double-stranded iRNA agent, or siRNA agent, (e.g., a precursor, e.g., a larger iRNA agent which can be processed into a siRNA agent, or a DNA which encodes an iRNA agent, e.g., a double-stranded iRNA agent, or siRNA agent, or precursor thereof) described herein can be formulated for controlled, e.g., slow release. Controlled release can be achieved by disposing the iRNA within a structure or substance which impedes its release. E.g., iRNA can be disposed within a porous matrix or in an erodable matrix, either of which allow release of the iRNA over a period of time.

Polymeric particles, e.g., polymeric in microparticles can be used as a sustained-release reservoir of iRNA that is taken up by cells only released from the microparticle through biodegradation. The polymeric particles in this embodiment should therefore be large enough to preclude phagocytosis (e.g., larger than 10 μm or larger than 20 μm). Such particles can be produced by the same methods to make smaller particles, but with less vigorous mixing of the first and second emulsions. That is to say, a lower homogenization speed, vortex mixing speed, or sonication setting can be used to obtain particles having a diameter around 100 μm rather than 10 μm. The time of mixing also can be altered.

Larger microparticles can be formulated as a suspension, a powder, or an implantable solid, to be delivered by intramuscular, subcutaneous, intradermal, intravenous, or intraperitoneal injection; via inhalation (intranasal or intrapulmonary); orally; or by implantation. These particles are useful for delivery of any iRNA when slow release over a relatively long term is desired. The rate of degradation, and consequently of release, varies with the polymeric formulation.

Microparticles may include pores, voids, hollows, defects or other interstitial spaces that allow the fluid suspension medium to freely permeate or perfuse the particulate boundary. For example, the perforated microstructures can be used to form hollow, porous spray dried microspheres.

Polymeric particles containing iRNA (e.g., a siRNA) can be made using a double emulsion technique, for instance. First, the polymer is dissolved in an organic solvent. A polymer may be polylactic-co-glycolic acid (PLGA), with a lactic/glycolic acid weight ratio of 65:35, 50:50, or 75:25. Next, a sample of nucleic acid suspended in aqueous solution is added to the polymer solution and the two solutions are mixed to form a first emulsion. The solutions can be mixed by vortexing or shaking, and in the mixture can be sonicated. Any method by which the nucleic acid receives the least amount of damage in the form of nicking, shearing, or degradation, while still allowing the formation of an appropriate emulsion is possible. For example, acceptable results can be obtained with a Vibra-cell model VC-250 sonicator with a ⅛″ microtip probe, at setting #3.

### Routes of Delivery

For ease of exposition the formulations, compositions and methods in this section are discussed largely with regard to unmodified iRNA agents. It may be understood, however, that these formulations, compositions and methods can be practiced with other iRNA agents, e.g., modified iRNA agents, and such practice is within the invention. A composition that includes a iRNA can be delivered to a subject by a variety of routes. Exemplary routes include: intravenous, topical, rectal, anal, vaginal, nasal, pulmonary, ocular.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

### Synthetic Methods

The invention is further illustrated by the following examples, which should not be construed as further limiting. The contents of all references, pending patent applications and published patents, cited throughout this application are hereby expressly incorporated by reference. The multifunction copolymers of the invention can be prepared by the following synthetic schemes.

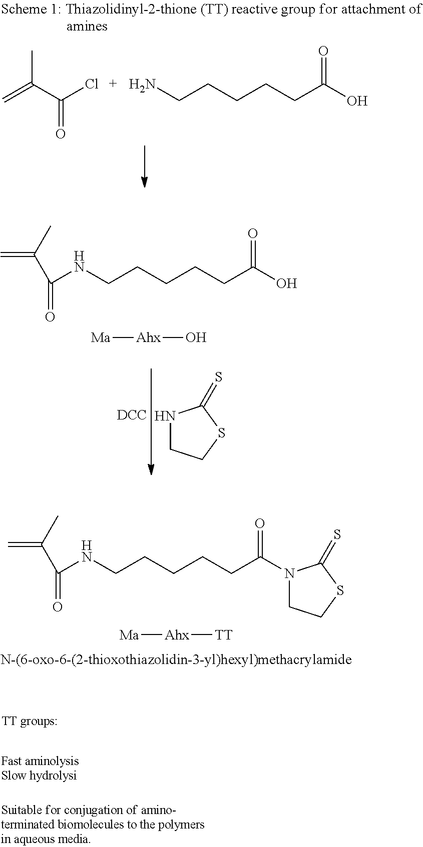

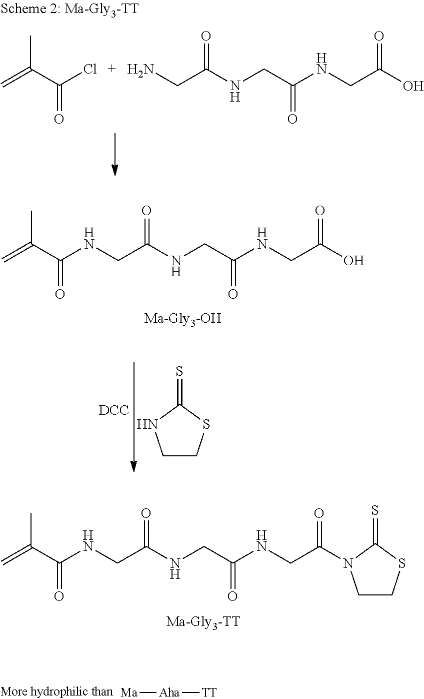

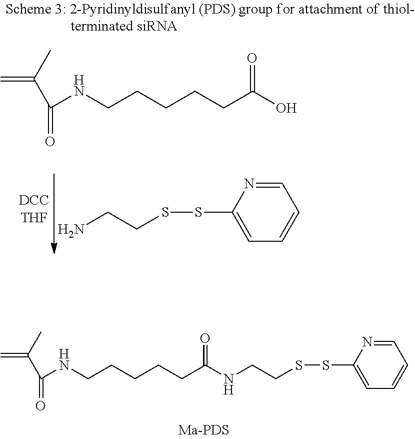 [SMILES: C=C(C)C(=O)NCCCCCC(=O)O ; NCCSSc1ccccn1 ; C=C(C)C(=O)NCCCCCC(=O)NCCSSc1ccccn1]

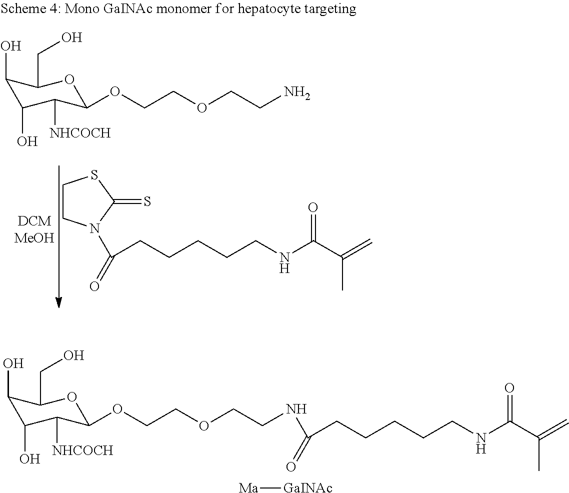

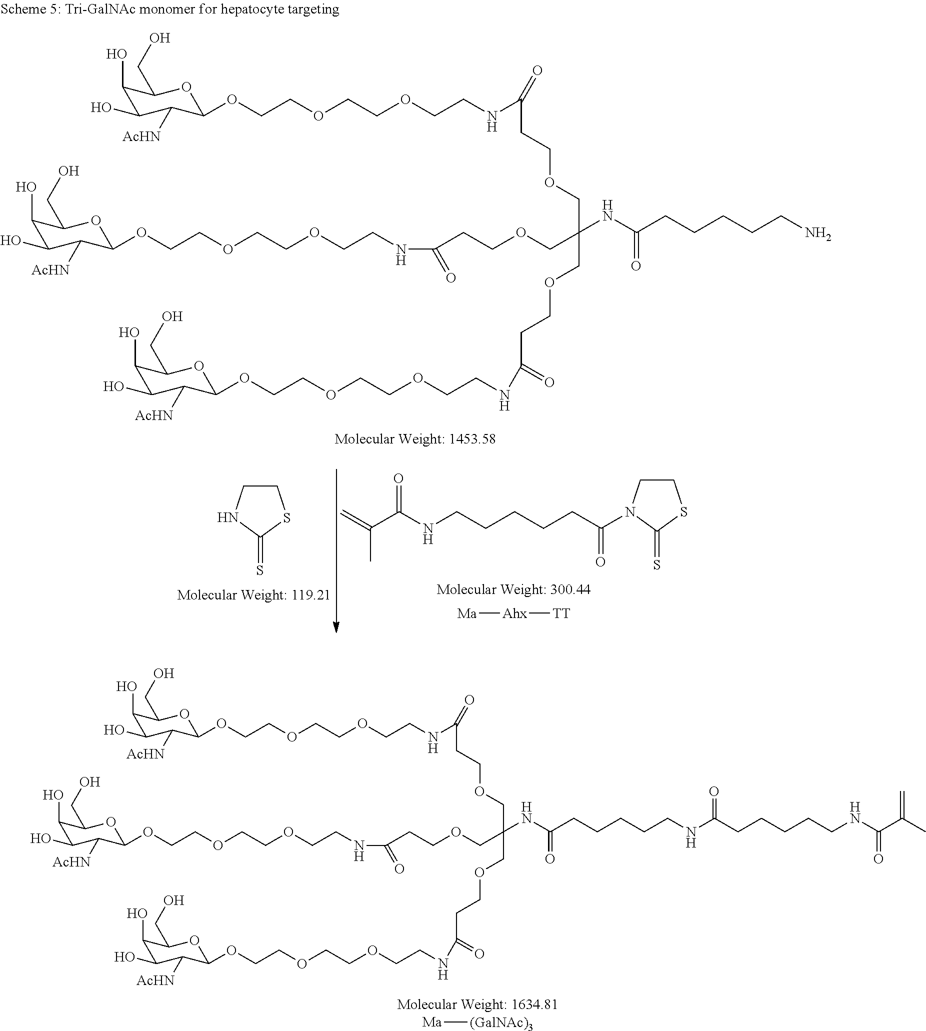

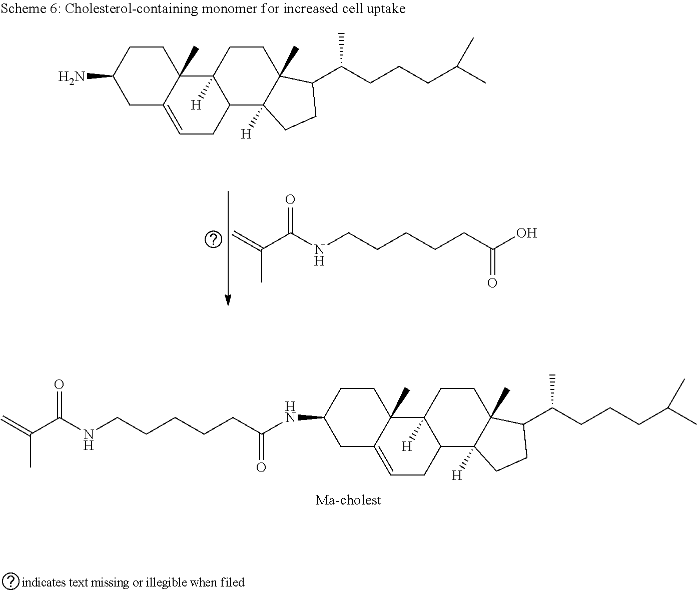

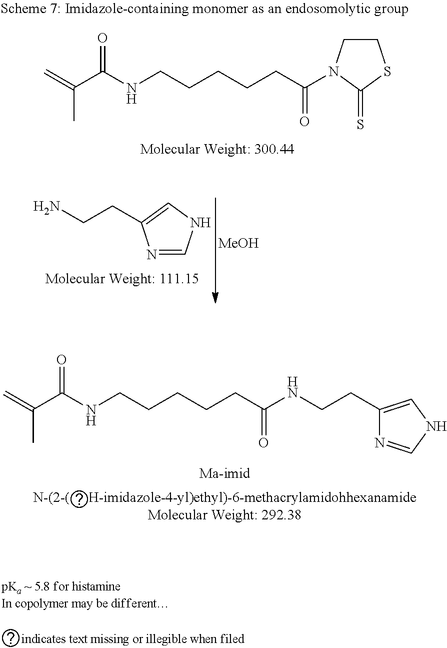 [SMILES: "MeOH" ; C=C(C)C(=O)NCCCCCC(=O)N1CCSC1=S ; NCCc1c[nH]cn1 ; C=C(C)C(=O)NCCCCCC(=O)NCCc1c[nH]cn1]

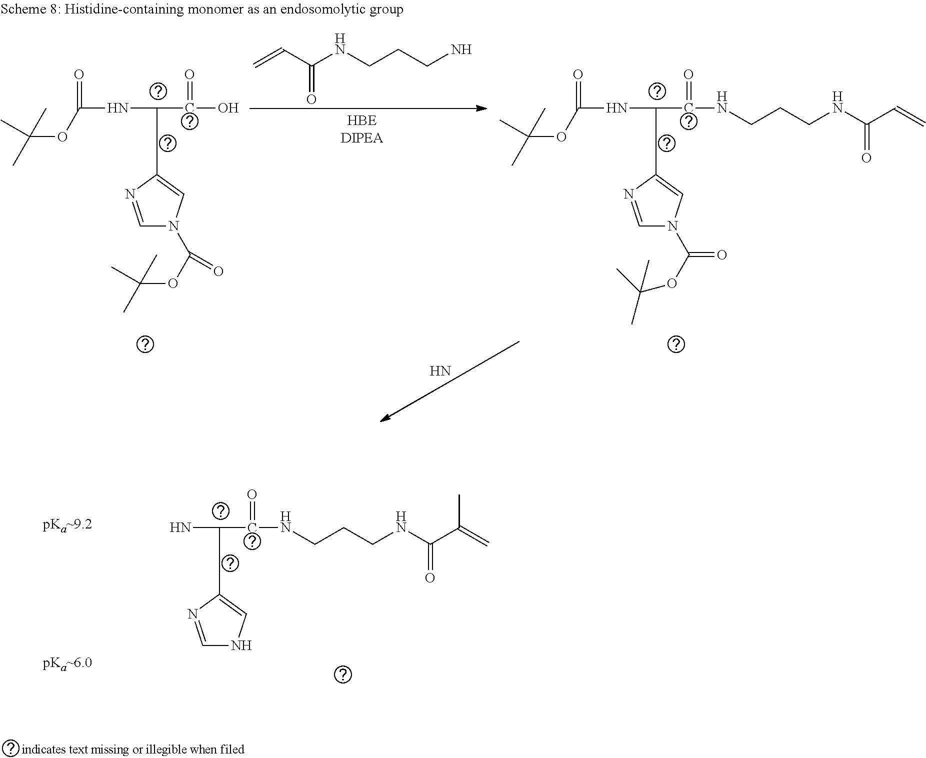

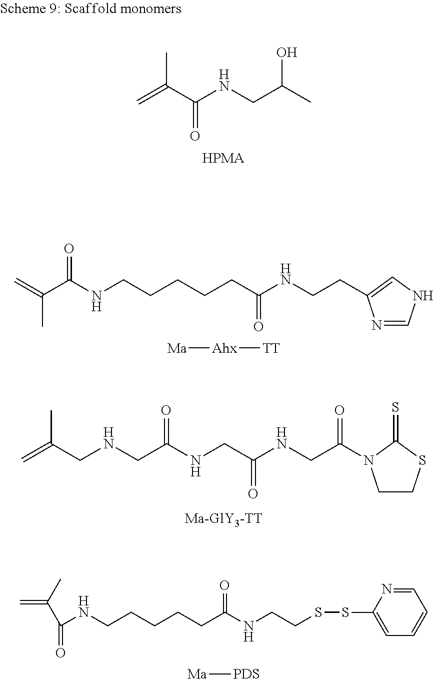

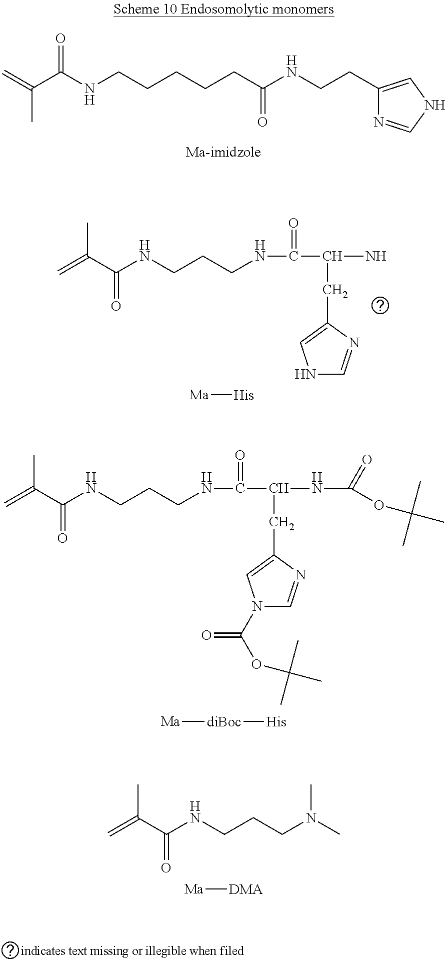

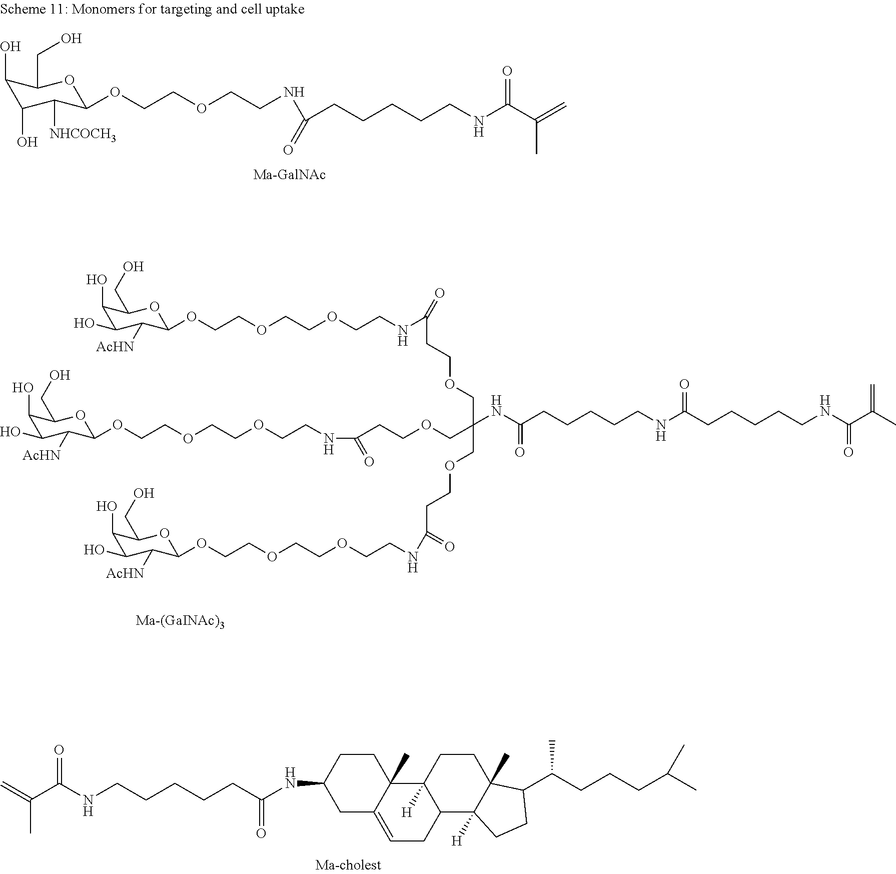

### Copolymers—First Generation

All copolymers were prepared by solution radical copolymerization in
DMSO at 60° C. using AIBN (2 wt %) as initiator and 15 wt %
of monomers.

| Polymer | | | | PDS | | | TT |
|---|---|---|---|---|---|---|---|
| (MP) | Composition | HPMA | PDS | (found) | GalNAc | TT | (found) |
| 5 | HPMA-TT | 95 | | | | 5 | 3.8 |
| 9 | HPMA-TT | 82 | | | | 18 | 18.3 |
| 13-1 | HPMA-TT- | 80 | 10 | 4.4 | | 10 | 9.9 |
| | PDS | | | | | | |
| 13-2 | HPMA-PDS- | 85 | 10 | 6.4 | 5 | | |
| | Gal | | | | | | |
| 13-3 | HPMA-TT- | 85 | | | 5 | 10 | 6.8 |
| | Gal | | | | | | |

### Copolymers—Second Generation

All copolymers were prepared by solution radical copolymerization in DMSO at 60° C. using
AIBN (1 wt %) as initiator and 15 wt % of monomers.

| Polymer | | | | PDS | | | | | | | TT |
|---|---|---|---|---|---|---|---|---|---|---|---|
| (MP) | Composition | HPMA | PDS | (found) | (GalNAc)3 | Histidine | imidazol | cholest | DMAP | TT | (found) |
| 17 | HPMA-Gal3-DMAP-TT | 60 | | | 5 | | | | 30 | 5 | 1.9 |
| 18 | HPMA-Gal3-TT-imid | 60 | | | 5 | 0 | 30 | | | 5 | 0 |
| 19c | HPMA-Gal3-PDS-imid | 60 | 5 | 2.9 | 5 | 0 | 30 | | | | |
| 23c | HPMA-Gal3-PDS-imid | 50 | 15 | 4 | 5 | 0 | 30 | | | | |
| 24 | HPMA-Gal3-PDS-His | 50 | 15 | 10 | 5 | 30 | | | | | |
| 26 | HPMA-Gal3-TT-His | 50 | | | 5 | 30 | | | | 10 | 0 |
| 27 | HPMA-Gal3-PDS-DMAP | 50 | 15 | 0 | 5 | 0 | 0 | 0 | 30 | | |
| 28b | HPMA-Gal3-PDS-His-cholest | 52 | 10 | 8.8 | 5 | 30 | 0 | 3 | | | |
| 29 | HPMA-Gal3-PDS | 80 | 15 | 7.3 | 5 | 0 | 0 | 0 | | | |
| 30a, c | HPMA-Gal3-PDS-imid- | 52 | 10 | 2.5 | 5 | 0 | 30 | 3 | | | |
| | cholest | | | | | | | | | | |
| 31 | HPMA-Gal3-PDS-imid | 60 | 15 | 0 | 5 | 0 | 30 | 0 | | | |
| 32a | HPMA-Gal3-PDS-His-cholest | 52 | 10 | 6.3 | 5 | 30 | | 3 | | | |
| 33a | HPMA-PDS-His-cholest | 57 | 10 | 8.1 | 0 | 30 | | 3 | | | |
| 36 | HPMA-Gal3-TT-diBocHis | 55 | | | 5 | 30 | | | | 10 | 0 |
| 37 | HPMA-TT-diBocHis | 60 | | | 0 | 30 | | | | 10 | 0 |

aPolymerized in methanol at 60° C., 14 hrs, 0.8% wt. of AIBN as initiator.
bInsoluble part was removed by filtration after polymerization.
cSolution of HCl in dioxane (4M, 0.125 ml) was added to polymerization mixture to protonate the imidazole monomer. In all other cases PDS groups were lost during polymerization.

### EQUIVALENTS

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

What is claimed is:

1. A multifunctional copolymer of formula (I): 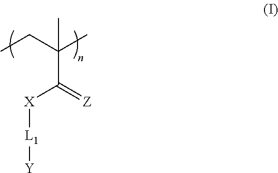
wherein:
Y is a nucleic acid or a ligand;
L₁ is a straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R′)C(O)O, OC(O)NR′, CH(Q), phosphorus containing linkage, aryl, heteroaryl, heterocyclic, or cycloalkyl;
R′ is hydrogen, acyl, aliphatic or substituted aliphatic;
Q is selected from the group consisting of OR₁₀, COR₁₀, CO₂R₁₀, 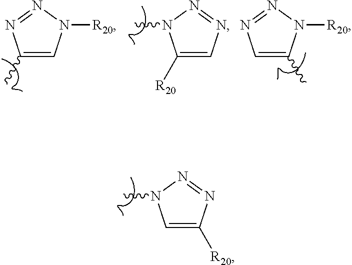
NR₂₀R₃₀, CONR₂₀R₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic;
R₂₀ and R₃₀ for each occurrence are independently selected from the group consisting of hydrogen, acyl, aliphatic or substituted aliphatic, aryl, heteroaryl, heterocyclic, OR₁₀, COR₁₀, CO₂R₁₀, and NR₁₀R₁₀′; or R₂₀ and R₃₀ are taken together to form a heterocyclic ring;
R₄₀ and R₅₀ for each occurrence are independently selected from the group consisting of is hydrogen, acyl, aliphatic or substituted aliphatic, aryl, heteroaryl, heterocyclic, OR₁₀, COR₁₀, CO₂R₁₀, and NR₁₀R₁₀′;
R₁₀ and R₁₀′ are independently hydrogen, aliphatic, substituted aliphatic, aryl, heteroaryl, or heterocyclic;
X is absent, O, or N(R′);
Z is O, S or NR′; and
n is an integer ranging from 5 to 20,000;
provided that at least one Y substituent is a nucleic acid, at least two Y substituents are ligands, and at least two of the ligands represent different compounds.

2. The multifunctional copolymer of claim 1, represented by formula (II): 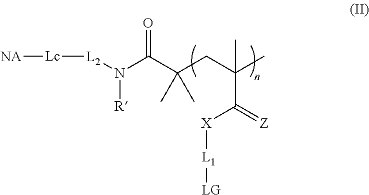
wherein:
NA is a nucleic acid;
Lc is a cleavable linker;
L₁ and L₂ are independently straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R′)C(O)O, OC(O)NR′, CH(Q), phosphorus containing linkage, aryl, heteroaryl, heterocyclic, or cycloalkyl;
R′ is hydrogen, acyl, aliphatic or substituted aliphatic;
Q is selected from the group consisting of OR₁₀, COR₁₀, CO₂R₁₀, 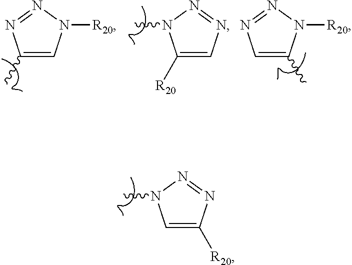
NR₂₀R₃₀, CONR₂OR₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic,
R₂₀ and R₃₀ for each occurrence are independently selected from the group consisting of hydrogen, acyl, aliphatic or substituted aliphatic, aryl, heteroaryl, heterocyclic, OR₁₀, COR₁₀, CO₂R₁₀, and NR₁₀R₁₀′; or R₂₀ and R₃₀ are taken together to form a heterocyclic ring;
R₄₀ and R₅₀ for each occurrence are independently selected from the group consisting of hydrogen, acyl, aliphatic or substituted aliphatic, aryl, heteroaryl, heterocyclic, OR₁₀, COR₁₀, CO₂R₁₀, and NR₁₀R₁₀'_{;}
R₁₀ and R₁₀′ are independently hydrogen, aliphatic, substituted aliphatic, aryl, heteroaryl, or heterocyclic;
X is absent, O, or N(R′);
Z is O, S or NR′;
n is an integer ranging from 5 to 20,000; and
LG is a ligand;
provided that at least two of the ligands represent different compounds.

3. The multifunctional copolymer of claim 2, represented by formula (III): 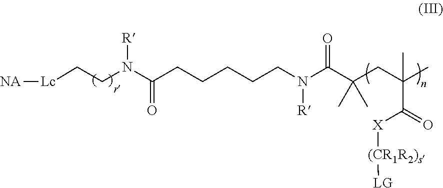
wherein:
NA is a nucleic acid;
Lc is a cleavable linker;
X is absent, O, or N(R′);
n is an integer ranging from 5 to 20,000;
s′ is an integer ranging from 1-20;
r′ is an integer ranging from 1-10;
R′ is independently for each occurrence hydrogen, acyl, aliphatic or substituted aliphatic;
R₁ and R₂ are each independently hydrogen or C₁-C₆ alkyl; and
LG is a ligand.

4. The multifunctional copolymer of claim 3, represented by formula (IV): 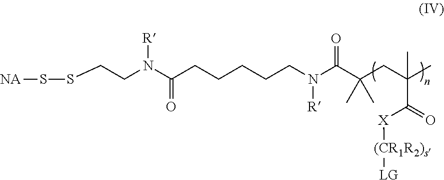
wherein:
NA is a nucleic acid;
X is absent, O, or N(R′);
R′ is independently for each occurrence hydrogen, acyl, aliphatic or substituted aliphatic;
n is an integer ranging from 5 to 20,000;
s′ is an integer ranging from 1-20;
R₁ and R₂ are each independently hydrogen or C₁-C₆ alkyl; and
and LG is a ligand.

5. A multifunctional copolymer of formula (V): 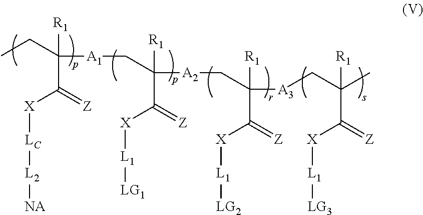
wherein:
NA is a nucleic acid;
each R₁ is independently hydrogen or C₁-C₆ alkyl;
A₁, A₂ and A₃ are either absent or a cleavable linker;
p, q, r, and s are each independently an integer ranging from 1 to 15,000;
Lc is a cleavable linker;
L₁ and L₂ are independently for each occurrence straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R′)C(O)O, OC(O)NR′, CH(Q), phosphorus containing linkage, aryl, heteroaryl, heterocyclic, or cycloalkyl;
R′ is hydrogen, acyl, aliphatic or substituted aliphatic;
Q is selected from the group consisting of OR₁₀, COR₁₀, CO₂R₁₀, 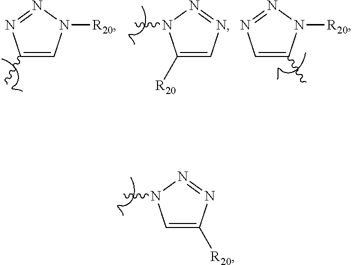
NR₂₀R₃₀, CONR₂OR₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic;
R₂₀ and R₃₀ for each occurrence are independently selected from the group consisting of hydrogen, acyl, aliphatic or substituted aliphatic, aryl, heteroaryl, heterocyclic, OR₁₀, COR₁₀, CO₂R₁₀, and NR₁₀R₁₀′; or R₂₀ and R₃₀ are taken together to form a heterocyclic ring;
R₄₀ and R₅₀ for each occurrence are independently selected from the group consisting of hydrogen, acyl, aliphatic or substituted aliphatic, aryl, heteroaryl, heterocyclic, OR₁₀, COR₁₀, CO₂R₁₀, and NR₁₀R₁₀′;
R₁₀ and R₁₀′ are independently hydrogen, aliphatic, substituted aliphatic, aryl, heteroaryl, or heterocyclic;
X is absent, O, or N(R′);
Z is O, S or NR′; and
LG₁, LG₂ and LG₃ are each independently selected from the group consisting of an endosomolytic ligand, a targeting ligand, and a PK modulator ligand.

6. The multifunctional copolymer of claim 5, wherein A₁, A₂ and A₃ are absent or independently selected from the group consisting of ester, disulfide, acetal, ketal, and hydrazone.

7. The multifunctional copolymer of claim 5, represented by formula (VI): 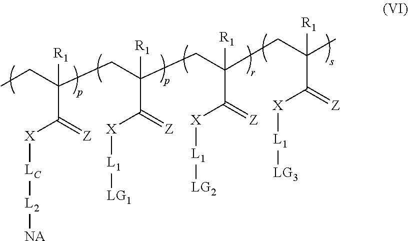
wherein:
NA is a nucleic acid;
each R₁ is independently hydrogen or C₁-C₆ alkyl;
p, q, r, and s are each independently an integer ranging from 1 to 15,000;
Lc is a cleavable linker;
L₁ and L₂ are independently for each occurrence straight- or branched-, substituted or unsubstituted alkyl, substituted or unsubstituted alkenyl, substituted or unsubstituted alkynyl, of which one or more methylenes can be interrupted by O, S, S(O), SO₂, N(R′), C(O), N(R′)C(O)O, OC(O)NR′, CH(Q), phosphorus containing linkage, aryl, heteroaryl, heterocyclic, or cycloalkyl;
R′ is hydrogen, acyl, aliphatic or substituted aliphatic;
Q is selected from the group consisting of OR₁₀, COR₁₀, CO₂R₁₀, 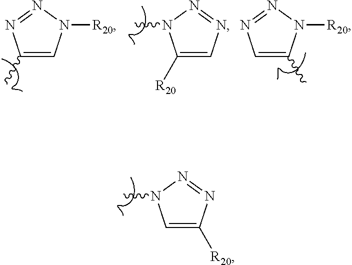
NR₂₀R₃₀, CONR₂OR₃₀, CON(H)NR₂₀R₃₀, ONR₂₀R₃₀, CON(H)N═CR₄₀R₅₀, N(R₂₀)C(═NR₃₀)NR₂₀R₃₀, N(R₂₀)C(O)NR₂₀R₃₀, N(R₂₀)C(S)NR₂₀R₃₀, OC(O)NR₂₀R₃₀, SC(O)NR₂₀R₃₀, N(R₂₀)C(S)OR₁₀, N(R₂₀)C(O)OR₁₀, N(R₂₀)C(O)SR₁₀, N(R₂₀)N═CR₄₀R₅₀, ON═CR₄₀R₅₀, SO₂R₁₀, SOR₁₀, SR₁₀ and substituted or unsubstituted heterocyclic;
R₂₀ and R₃₀ for each occurrence are independently selected from the group consisting of hydrogen, acyl, aliphatic or substituted aliphatic, aryl, heteroaryl, heterocyclic, OR₁₀, COR₁₀, CO₂R₁₀, and NR₁₀R₁₀′; or R₂₀ and R₃₀ are taken together to form a heterocyclic ring;
R₄₀ and R₅₀ for each occurrence are independently selected from the group consisting of hydrogen, acyl, aliphatic or substituted aliphatic, aryl, heteroaryl, heterocyclic, OR₁₀, COR₁₀, CO₂R₁₀, and NR₁₀R₁₀′;
R₁₀ and R₁₀′ are independently hydrogen, aliphatic, substituted aliphatic, aryl, heteroaryl, or heterocyclic;
X is absent, O, or N(R′);
Z is O, S or NR′; and
LG₁, LG₂ and LG₃ are each independently selected from the group consisting of an endosomolytic ligand, a targeting ligand, and a PK modulator ligand.

8. The multifunctional copolymer of claim 5, wherein the endosomolytic ligand is selected from the group consisting of imidazoles, poly or oligoimidazoles, linear or brached polyethyleneimines (PEIs), linear and branched polyamines, cationic linear and branched polyamines, polycarboxylates, polycations, masked oligo or poly cations or anions, acetals, polyacetals, ketals, polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, and natural and synthetic fusogenic lipids.

9. The multifunctional copolymer of claim 5, wherein the endosomolytic ligand is a polyanionic peptide or a polyanionic peptidomimetic.

10. The multifunctional copolymer of claim 5, wherein the endosomolytic ligand is selected from the group consisting of GALA, EALA, INF-7, Inf HA-2, diINF-7, diINF3, GLF, GALA-INF3, INF-5, JTS-1, ppTG1, ppTG20, KALA, HA, melittin, and histinde-rich peptide CHK₆HC.

11. The multifunctional copolymer of claim 5, wherein the targeting ligand is selected from the group consisting of an antibody, a ligand-binding portion of a receptor, a ligand for a receptor, an aptamer, D-galactose, N-acetyl-D-galactose (GalNAc), multivalent N-acetyl-D-galactose, D-mannose, cholesterol, a fatty acid, a lipoprotein, folate, thyrotropin, melanotropin, surfactant protein A, mucin, carbohydrate, multivalent lactose, multivalent galactose, N-acetyl-galactosamine, N-acetyl-gulucosamine, multivalent mannose, multivalent fucose, glycosylated polyaminoacids, transferrin, bisphosphonate, polyglutamate, polyaspartate, a lipophilic moiety that enhance plasma protein binding, a steroid, bile acid, vitamin B₁₂, biotin, an RGD peptide, an RGD peptide mimetic, ibuprofen, naproxen, aspirin, folate, and analogs and derivatives thereof.

12. The multifunctional copolymer of claim 11, wherein the targeting ligand is selected from the group consisting of D-galactose, N-acetyl-D-galactose (GalNAc), multivalent N-acetyl-D-galactose, cholesterol, folate, and analogs and derivates thereof.

13. The multifunctional copolymer of claim 5, wherein the nucleic acid is selected from the group consisting of an iRNA agent, an antisense oligonucleotide, an antagomir, an activating RNA, a decoy oligonucleotide, an aptamer, and a ribozyme.

14. The multifunctional copolymer of claim 5, wherein the nucleic acid contains at least one sugar modification.

15. The multifunctional copolymer of claim 14, wherein said sugar modification is a 2′-modification.

16. The multifunctional copolymer of claim 15, wherein said 2′-modificaiton is selected from the group consisting of 2′-O-Me (2′-O-methyl), 2′-O-MOE (2′-O-methoxyethyl), 2′-F, 2′-O-[2-(methylamino)-2-oxoethyl] (2′-O-NMA), 2′—NH₂, 2′-O-amine, 2′-SH, 2′-S-alkyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), and 2′-O-dimethylaminoethyloxyethyl (2′-O-DMAEOE).

17. The multifunctional copolymer of claim 5, wherein the nucleic acid contains at least one backbone modification.

18. The multifunctional copolymer of claim 17, wherein said backbone modification is selected from the group consisting of phosophorothioate, phosphorodithioate, phosphoramidate, phosphonate, alkylphosphonate, siloxane, carbonate, carboxymethyl, carbamate, amide, thioether, ethylene oxide linker, sulfonate, sulfonamide, thioformacetal, formacetal, oxime, methyleneimino, methyleneaminocarbonyl, methylenemethylimino (MMI), methylenehydrazo, methylenedimethylhydrazo (MDH), and methyleneoxymethylimino.

19. The multifunctional copolymer of claim 5, wherein the nucleic acid down regulates the expression of a target gene.

20. The multifunctional copolymer of claim 17, wherein the nucleic acid down regulates the expression of a target gene through an RNA interference mechanism.

21. The multifunctional copolymer of claim 5, wherein the nucleic acid is a single-stranded oligonucleotide.

22. The multifunctional copolymer of claim 5, wherein the nucleic acid is a double-stranded oligonucleotide.

23. The multifunctional copolymer of claim 5, wherein Lc is a redox-cleavable linker.

24. The multifunctional copolymer of claim 5, wherein Lc comprises at least one pH-sensitive component.

25. A method of delivering a multifunctional copolymer to a cell, the method comprising (a) contacting a cell with the multifunctional copolymer of claim 5; and (b) allowing the cell to internalize the multifunctional copolymer.

26. The method of claim 25, wherein at least one of LG₁, LG₂, and LG₃ is a targeting ligand.

27. The method of claim 26, wherein the targeting ligand provides sufficient permeability and retention to allow the nucleic acid to accumulate in the cell.

28. A method of inhibiting the expression of one or more genes, comprising contacting one or more cells with an effective amount of the multifunctional copolymer of claim 5, wherein the effective amount is an amount that suppresses the expression of the one or more genes.
